(19) United States
(12) Patent Application Publication
(10) Pub. No.: US 2010/0286250 A1
(43) Pub. Date: Nov. 11, 2010

(54) FLUORINATED LIPIDS AND METHODS OF USE

(75) Inventors: Venkateshwarlu Kalsani, Watertown, MA (US); Laila Dafik, Medford, MA (US); Krishna Kumar, Cambridge, MA (US); Tarakkad Subrahmanian Krishnaji, Medford, MA (US)

Correspondence Address: CHOATE, HALL & STEWART LLP, TWO INTERNATIONAL PLACE, BOSTON, MA, 02110, US

(73) Assignee: TUFTS UNIVERSITY, Medford, MA (US)

(21) Appl. No.: 12/680,024

(22) PCT Filed: Sep. 26, 2008
(86) PCT No.: PCT/US08/78030

§ 371 Date: Jun. 28, 2010

Related U.S. Application Data
(60) Provisional application No. 60/975,345, filed on Sep. 26, 2007.

(51) Int. Cl. A61K 48/00; C07H 21/02; C12N 5/00

(52) U.S. Cl. 514/44 R; 536/23.1; 435/375

(57) ABSTRACT

The invention provides methods and compounds for delivering agents to cells. The compounds can include a fluorinated lipid, a linker, and an agent for delivery to a cell.

### CROSS-REFERENCE TO RELATED APPLICATIONS

This application claims the benefit of priority of U.S. Ser. No. 60/975,345, filed Sep. 26, 2007, the contents of which are hereby incorporated by reference in their entirety for all purposes.

### BACKGROUND OF THE INVENTION

The plasma membrane enveloping mammalian cells serves a crucial gatekeeping function by careful regulation of the influx and exodus of molecules. Only small (<1 kDa), hydrophobic molecules pass through the membrane by passive diffusion. All other types of molecules have to confront the impervious and selective membrane barrier to gain entry. Strategies to deliver macromolecules into living cells have tremendous potential in therapeutic and imaging applications (Smith and van de Waterbeemd, Curr. Opin. Chem. Biol. 3:373, 1999; Doerr, Nat. Meth. 3:770, 2006). Conjugates with amphipathic, hydrophobic or cationic polymers, and carbon nanotubes have been deployed. Noncovalent assemblies of cationic lipids and macromolecules, and liposomes have also been used. Existing cationic lipid delivery systems are disruptive to cell membranes, which causes cellular cytotoxicity, and efficiency of delivery with these agents is highly variable. There is a need for new classes of molecules that are non-toxic, efficient, and that have reasonable half-lives inside cells to be general in their applicability. Such molecules can potentially be used to deliver agents such as nucleic acids, e.g., for use in chemo- and genetic therapy or imaging.

### SUMMARY OF THE INVENTION

The present invention provides methods and compositions for introducing agents such as nucleic acids, peptides, and small molecules into cells. The invention is based, in part, on the discovery of new classes of fluorinated phospholipids that are non-toxic and mediate highly efficient delivery of agents into living cells. The fluorinated phospolipid compounds described herein have increased hydrophobicity and affinity for membranes relative to non-fluorinated lipids. The fluorinated phospholipid compounds described herein can enter cells by mechanisms that include endocytosis. In some embodiments, fluorinated phospholipid compounds described herein have properties that deliver agents to the cytoplasm and/or membranes of living cells, while avoiding or minimizing delivery of the agents into cell nuclei, in contrast to other types of lipophilic delivery agents.

Fluorinated phospholipids can be covalently linked to an agent for delivery to a cell. In some embodiments, the agent for delivery to a cell is a biologically active molecule. In some embodiments, the agent to which a lipid is attached binds to a biologically active molecule by a noncovalent interaction, and the agent can mediate delivery of the biologically active molecule to the cell indirectly. In certain embodiments, a fluorinated phospholipid is connected to a linker, e.g., a labile linker, e.g., a linker that is labile once inside a cell or subcellular compartment, e.g., a linker that is cleaved under acidic conditions, or in the presence of an enzyme (e.g., a lipase- or protease-sensitive linker). Delivery of agents via cleavable linkers permits efficient delivery to cells and release of an agent intracellularly. In certain embodiments, provided fluorinated phospholipids permit delivery of a therapeutic agent in the presence of serum.

Accordingly, in one aspect, the invention features a compound that includes a non-cationic phospholipid, a linker, and an agent for delivery to a cell, wherein at least one hydrocarbon chain of the phospholipid is fluorinated, and wherein the phospholipid, the linker, and the agent are covalently linked. In some embodiments, the compound comprises the following formula:

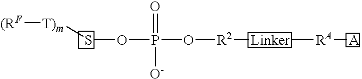

wherein:

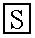

is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{A} is a covalent bond or an optionally substituted moiety derived from conjugating an optionally substituted thiol-reactive, amine-reactive, or hydroxyl-reactive moiety with a thiol, amine, or hydroxyl group of the agent;

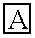

is a therapeutic agent;
each occurrence of n is an integer from 0 to 30, inclusive, wherein at least one occurrence of n is non-zero; and
m is an integer from 1 to 2, inclusive, wherein m is 1 when

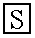

is a covalent bond.

According to one aspect, the present invention provides a non-cationic fluorinated phospholipid of the formula:

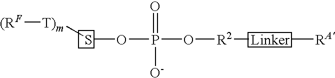

wherein:

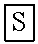

is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{A′} is hydrogen or an optionally substituted thiol-reactive, amine-reactive, or hydroxyl-reactive moiety;
each occurrence of n is an integer from 0 to 30, inclusive, wherein at least one occurrence of n is non-zero; and

m is an integer from 1 to 2, inclusive, wherein m is 1 when

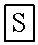

is a covalent bond.

According to one aspect, the present invention provides compound comprising a non-cationic phospholipid, wherein at least one hydrocarbon chain of the phospolipid is fluorinated and the compound is of the formula:

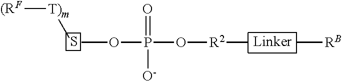

wherein:

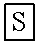

is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{B} is an optionally substituted moiety capable of forming a non-covalent interaction with a therapeutic agent;
each occurrence of n is an integer from 0 to 30, inclusive, wherein at least one occurrence of n is non-zero; and
m is an integer from 1 to 2, inclusive, wherein m is 1 when

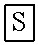

is a covalent bond.

According to one aspect, the present invention provides a composition comprising:

a) a non-cationic phospholipid, wherein at least one hydrocarbon chain of the phospolipid is fluorinated and the compound is of the formula:

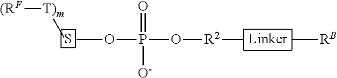

wherein:

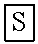

is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{B} is an optionally substituted moiety capable of forming a non-covalent interaction with a therapeutic agent;
each occurrence of n is an integer from 0 to 30, inclusive, wherein at least one occurrence of n is non-zero; and
m is an integer from 1 to 2, inclusive, wherein m is 1 when

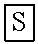

is a covalent bond; and
b) a therapeutic agent

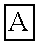

non-covalently linked to R^{B}.

According to one aspect, the present invention provides a method for delivering a therapeutic agent into a cell, the method comprising contacting a cell with a compound or composition as described herein.

According to one aspect, the present invention provides a kit for delivering a macromolecule into a cell, the kit comprising a compound, wherein the compound comprises a phospholipid covalently linked to a linker, wherein at least one hydrocarbon chain of the phospholipid is fluorinated, and wherein the linker comprises a reactive moiety. In certain embodiments, the kit comprises a phospholipid having the formula:

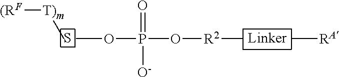

wherein:

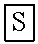

is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{A′} is hydrogen or an optionally substituted thiol-reactive, amine-reactive, or hydroxyl-reactive moiety;
each occurrence of n is an integer from 0 to 30, inclusive, wherein at least one occurrence of n is non-zero; and
m is an integer from 1 to 2, inclusive, wherein m is 1 when

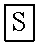

is a covalent bond.

According to one aspect, the present invention provides a method comprising the steps of:
a) providing a non-cationic fluorinated phospholipid of the formula:

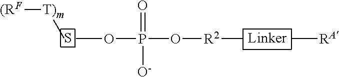

wherein:

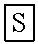

is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{A′} is hydrogen or an optionally substituted thiol-reactive, amine-reactive, or hydroxyl-reactive moiety;
each occurrence of n is an integer from 0 to 30, inclusive, wherein at least one occurrence of n is non-zero; and
m is an integer from 1 to 2, inclusive, wherein m is 1 when

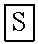

is a covalent bond; and
b) contacting the fluorinated phospholipid with an agent for delivery to a cell to form a compound of formula:

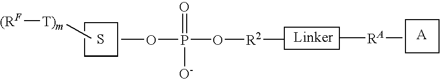

wherein:
   R^{A} is a covalent bond or an optionally substituted moiety derived from conjugating an optionally substituted thiol-reactive, amine-reactive, or hydroxyl-reactive moiety with a thiol, amine, or hydroxyl group of the agent; and

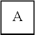

is a therapeutic agent.

### BRIEF DESCRIPTION OF THE DRAWINGS

FIG. 1 is a schematic diagram of exemplary phospholipid structures and synthesis from the corresponding H-phosphonate.

FIG. 2 is a bar graph depicting relative fluorescence from HeLa cells treated with 3 or the compound 2:AF under various conditions. Each bar in the set corresponds to the following conditions, in this order: −, 37° C., 4° C., +4 mM NaN₃, +0.30 M sucrose.

FIG. 3 is a set of confocal fluorescence microscopy images of HeLa cells treated with 3 (FIG. 3A, FIG. 3B) and with 2:AF (FIG. 3C, FIG. 3D). Propidium iodide was used to stain the nucleus. Box 40μ×40μ and the z dimension represents intensity. Image is one of 18 slices taken over a 1 μm distance.

FIG. 4 is a set of fluorescence microscopy images of HeLa cells treated with 2:AF and 3 at 100 μM (a,d), at 10 μM (b, e) and at 0 μM (c,f) respectively. In both cases DAPI was used to stain the nucleus (gray; a-f). Overlay images clearly show that material is excluded from the nucleus. Images were taken at the same exposure for different concentrations of the lipid constructs. Bar, 30 μm.

FIG. 5 is a set of graphs depicting results of flow cytometry of HeLa cells treated with 8 and 3 (FIG. 5A) and with 3 and 5 (FIG. 5B).

FIG. 6A is a schematic diagram of the synthesis of compound 6.

FIG. 6B is a schematic diagram of the synthesis and structural features of lipid conjugates 1-5.

FIG. 7 depicts positive mode ESI-MS spectrum and isotopic distribution (inset: lower lines are calculated and upper lines are experimental) of 1 in CH₂Cl₂.

FIG. 8 depicts negative mode ESI-MS spectrum and isotopic distribution (inset: lower lines are calculated and upper lines are experimental) of 2 in CH₂Cl₂.

FIG. 9 depicts negative mode ESI-MS spectrum and isotopic distribution (inset: lower lines are calculated and upper lines are experimental) of 3 in CH₂Cl₂.

FIG. 10 depicts negative mode ESI-MS spectrum and isotopic distribution (inset: lower lines are calculated and upper lines are experimental) of 4 in CH₂Cl₂.

FIG. 11 depicts Negative mode ESI-MS spectrum of 5 in CH₂Cl₂.

FIG. 12 is a set of graphs showing decrease of fluorescence from HeLa cells upon reduction with dithionite (FIG. 12A, Na₂S₂O₄) and exchange with biotin (FIG. 12B) after treatment with agents 3 and 2:AF. Cells were treated with compound 3 (FIG. 12A) or the 2:AF complex (FIG. 12B) for 2 h at 37° C., washed with PBS and then subjected to reduction by Na₂S₂O₄ (5 mM, 1 m) or exchange with biotin (20 eq., 1 h) followed by 2× wash with PBS. Fluorescence was recorded in a 96-well plate on a microtiter plate reader from 1×10⁵ cells in 100 μL. There was no change in fluorescence if cells in (FIG. 12A) were treated with biotin, or if cells in (FIG. 12B) were treated with Na₂S₂O₄. The results are an average of three independent experiments in six replicates. Error bars represent one S.D.

FIG. 13 is a set of fluorescence microscopy images of HeLa cells incubated with a) 100 μM 8 (FIG. 13A) and, b) 100 μM 3 (FIG. 13B) for 2 h at 37° C. After being washed twice with PBS at room temperature, cells were analyzed by fluorescence microscopy.

### DEFINITIONS

Agent for delivery to a cell: As used herein, the phrase “agent for delivery to a cell” refers to any substance that can be delivered to a cell (e.g., in vitro or in vivo, e.g., to a tissue, cell, or subcellular location). In some embodiments, the agent for delivery to a cell is a macromolecule such as a nucleic acid (e.g., RNA, DNA), a peptide (e.g., an antibody, growth factor, transcription factor, peptide hormone, or other peptide), carbohydrate, lipid, or other type of macromolecule. In some embodiments, an agent for delivery to a cell is a small molecule (e.g., a small molecule which is membrane impermeable when it is not associated with a compound described herein). In some embodiments, an agent for delivery to a cell is biologically active, e.g., when delivered to a cell, the agent has an effect on the cell (e.g., the agent binds to a molecule within the cell, and/or the agent has an effect on a biological function of the cell, and/or the agent causes a change in gene expression in the cell, etc.). In some embodiments, the agent for delivery to a cell is an agent that specifically binds to a macromolecule and, when associated with the macromolecule, mediates delivery of the macromolecule into the cell. One example of such an agent is biotin, which specifically binds to avidin, streptavidin, and derivatives thereof. Other examples include small molecule inhibitors of peptides.

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties are physically associated. In some embodiments, the moieties are attached to one another by one or more covalent bonds. In some embodiments, the moieties are attached to one another by a mechanism that involves specific (but non-covalent) binding (e.g. streptavidin/avidin interactions, antibody/antigen interactions, etc.). In some embodiments, a sufficient number of weaker interactions can provide sufficient stability for moieties to remain physically associated. In some embodiments, the moieties are attached by a reversible interaction (e.g., a covalent interaction which is stable under one set of conditions and labile under another set of conditions such as exposure to acidic pH or to an enzyme).

Inhibit expression of a gene: As used herein, the phrase “inhibit expression of a gene” means to cause a reduction in the amount of an expression product of the gene. The expression product can be an RNA transcribed from the gene (e.g. an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., a test tube or reaction vessel, a cell culture, etc., rather than within a multi-cellular organism.

In vivo: As used herein, the term “in vivo” refers to events that occur within a multi-cellular organism such as a non-human animal.

microRNA (miRNA): As used herein, the term “microRNA” or “miRNA” refers to an RNAi agent that is approximately 21 nucleotides (nt)-23 nt in length. miRNAs can range between 18 nt-26 nt in length. Typically, miRNAs are single-stranded. However, in some embodiments, miRNAs may be at least partially double-stranded. In certain embodiments, miRNAs may comprise an RNA duplex (referred to herein as a “duplex region”) and may optionally further comprises one or two single-stranded overhangs. In some embodiments, an RNAi agent comprises a duplex region ranging from 15 by to 29 by in length and optionally further comprising one or two single-stranded overhangs. An miRNA may be formed from two RNA molecules that hybridize together, or may alternatively be generated from a single RNA molecule that includes a self-hybridizing portion. In general, free 5′ ends of miRNA molecules have phosphate groups, and free 3′ ends have hydroxyl groups. The duplex portion of an miRNA usually, but does not necessarily, comprise one or more bulges consisting of one or more unpaired nucleotides. One strand of an miRNA includes a portion that hybridizes with a target RNA. In certain embodiments, one strand of the miRNA is not precisely complementary with a region of the target RNA, meaning that the miRNA hybridizes to the target RNA with one or more mismatches. In some embodiments, one strand of the miRNA is precisely complementary with a region of the target RNA, meaning that the miRNA hybridizes to the target RNA with no mismatches. Typically, miRNAs are thought to mediate inhibition of gene expression by inhibiting translation of target transcripts. However, in some embodiments, miRNAs may mediate inhibition of gene expression by causing degradation of target transcripts.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, e.g., analogs having other than a phosphodiester backbone, an analogs that include a thiolated residue. For example, the so-called “peptide nucleic acids,” which are known in the art and have peptide bonds instead of phosphodiester bonds in the backbone, are considered within the scope of the present invention. Nucleotide sequences that encode proteins and/or RNA may include introns. Nucleic acids can be purified from natural sources, produced using recombinant expression systems and optionally purified, chemically synthesized, etc. Where appropriate, e.g. in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, 4, 5, 6, 7, 8, 9, 10, or more residues. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Peptide: As used herein, the term “peptide” refers to a peptide, protein, or polypeptide (i.e., a string of at least two amino acids linked to one another by peptide bonds). Peptides may include moieties other than amino acids (e.g., may be glycoproteins, proteoglycans, etc.) and/or may be otherwise processed or modified. Those of ordinary skill in the art will appreciate that a “peptide” can be a complete polypeptide chain as produced by a cell (with or without a signal sequence), or can be a characteristic portion thereof. Those of ordinary skill will appreciate that a peptide can sometimes include more than one peptide chain, for example linked by one or more disulfide bonds or associated by other means. Peptides may contain L-amino acids, D-amino acids, or both and may contain any of a variety of amino acid modifications or analogs known in the art. Useful modifications include, e.g., terminal acetylation, amidation, etc. In some embodiments, peptides may comprise natural amino acids, non-natural amino acids, synthetic amino acids, and combinations thereof.

RNA interference (RNAi): As used herein, the term “RNA interference” or “RNAi” refers to sequence-specific inhibition of gene expression and/or reduction in target RNA levels mediated by an at least partly double-stranded RNA, which RNA comprises a portion that is substantially complementary to a target RNA. Typically, at least part of the substantially complementary portion is within the double stranded region of the RNA. In some embodiments, RNAi can occur via selective intracellular degradation of RNA. In some embodiments, RNAi can occur by translational repression. Agents that mediate RNAi (“RNAi agents”) include, for example, small interfering RNAs (siRNA), short hairpin RNAs (shRNA), and microRNAs (miRNA). In some embodiments, agents that mediate RNAi include one or more nucleotide analogs or modifications, having a structure characteristic of molecules that can mediate inhibition of gene expression through an RNAi mechanism. In some embodiments, RNAi agents mediate inhibition of gene expression by causing degradation of target transcripts. In some embodiments, RNAi agents mediate inhibition of gene expression by inhibiting translation of target transcripts. Generally, an RNAi agent includes a portion that is substantially complementary to a target RNA. In some embodiments, RNAi agents are at least partly double-stranded. In some embodiments, RNAi agents are single-stranded. In some embodiments, RNAi agents may be composed entirely of natural RNA nucleotides (i.e., adenine, guanine, cytosine, and uracil). In some embodiments, RNAi agents may include one or more non-natural RNA nucleotides (e.g. nucleotide analogs, DNA nucleotides, etc.). Inclusion of non-natural RNA nucleic acid residues may be used to make the RNAi agent more resistant to cellular degradation than RNA. In some embodiments, the term “RNAi agent” may refer to any RNA, RNA derivative, and/or nucleic acid encoding an RNA that induces an RNAi effect (e.g. degradation of target RNA and/or inhibition of translation). In some embodiments, an RNAi agent may comprise a blunt-ended (i.e., without overhangs) dsRNA that can act as a Dicer substrate. For example, such an RNAi agent may comprise a blunt-ended dsRNA which is >25 base pairs length, which may optionally be chemically modified to abrogate an immune response.

Short, interfering RNA (siRNA): As used herein, the term “short, interfering RNA” or “siRNA” refers to an RNAi agent comprising an RNA duplex (referred to herein as a “duplex region”) that is approximately 19 basepairs (bp) in length and optionally further comprises one or two single-stranded overhangs. In some embodiments, an RNAi agent comprises a duplex region ranging from 15 by to 29 by in length and optionally further comprising one or two single-stranded overhangs. An siRNA may be formed from two RNA molecules that hybridize together, or may alternatively be generated from a single RNA molecule that includes a self-hybridizing portion. The duplex portion of an siRNA may, but typically does not, comprise one or more bulges consisting of one or more unpaired nucleotides. One strand of an siRNA includes a portion that hybridizes with a target RNA. In certain embodiments, one strand of the siRNA is precisely complementary with a region of the target RNA, meaning that the siRNA hybridizes to the target RNA without a single mismatch. In some embodiments, one or more mismatches between the siRNA and the targeted portion of the target RNA may exist. In some embodiments in which perfect complementarity is not achieved, any mismatches are generally located at or near the siRNA termini. In some embodiments, siRNAs mediate inhibition of gene expression by causing degradation of target transcripts.

Short hairpin RNA (shRNA): As used herein, the term “short hairpin RNA” or “shRNA” refers to an RNAi agent comprising an RNA having at least two complementary portions hybridized or capable of hybridizing to form a double-stranded (duplex) structure sufficiently long to mediate RNAi (typically at least approximately 19 by in length), and at least one single-stranded portion, typically ranging between approximately 1 nucleotide (nt) and approximately 10 nt in length that forms a loop. In some embodiments, an shRNA comprises a duplex portion ranging from 15 by to 29 by in length and at least one single-stranded portion, typically ranging between approximately 1 nt and approximately 10 nt in length that forms a loop. The duplex portion may, but typically does not, comprise one or more bulges consisting of one or more unpaired nucleotides. In some embodiments, siRNAs mediate inhibition of gene expression by causing degradation of target transcripts. shRNAs are thought to be processed into siRNAs by the conserved cellular RNAi machinery. Thus shRNAs may be precursors of siRNAs. Regardless, siRNAs in general are capable of inhibiting expression of a target RNA, similar to siRNAs.

Small Molecule: In general, a “small molecule” is understood in the art to be an organic molecule that is less than about 5 kilodaltons (Kd) in size. In some embodiments, the small molecule is less than about 4 Kd, about 3 Kd, about 2 Kd, or about 1 Kd. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, small molecules are non-polymeric. In some embodiments, small molecules are not proteins, peptides, or amino acids. In some embodiments, small molecules are not nucleic acids or nucleotides. In some embodiments, small molecules are not saccharides or polysaccharides.

Specific binding: As used herein, the term “specific binding” refers to non-covalent physical association of a first and a second moiety wherein the association between the first and second moieties is at least 100 times as strong as the association of either moiety with most or all other moieties present in the environment in which binding occurs. Binding of two or more entities may be considered specific if the equilibrium dissociation constant, K_{d}, is 10⁻⁶M or less, 10⁻⁷ M or less, 10⁻³ M or less, or 10⁻⁹ M or less under the conditions employed, e.g. under physiological conditions such as those inside a cell or consistent with cell survival. Examples of specific binding interactions include antibody-antigen interactions, avidin-biotin interactions, hybridization between complementary nucleic acids, etc.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition.

Therapeutic agent: As used herein, the phrase “therapeutic agent” refers to any agent that, when administered to a subject, has a therapeutic effect and/or elicits a desired biological and/or pharmacological effect.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, chiral chromatography, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in inhibiting Sonic Hedgehog Protein-induced transcription. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term acyl as used herein refers to a moiety that includes a carbonyl group oro a group having the general formula —C(═O)R, where R is alkyl, alkenyl, alkynyl, aryl, carbocylic, heterocyclic, or aromatic heterocyclic. An example of an acyl group is acetyl.

The term aliphatic, as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

The term alkyl as used herein refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-12 carbon atoms. In another embodiment, the alkyl group employed contains 1-8 carbon atoms. In still other embodiments, the alkyl group contains 1-6 carbon atoms. In yet another embodiment, the alkyl group contains 1-4 carbons. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substituents.

In general, the terms aryl and heteroaryl, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substituents, i.e., the substituents recited for aliphatic moieties, or for other moieties as disclosed herein, resulting in the formation of a stable compound. In certain embodiments of the present invention, aryl refers to a mono- or bicyclic carbocyclic ring system having one or two aromatic rings including, but not limited to, phenyl, naphthyl, tetrahydronaphthyl, indanyl, indenyl, and the like. In certain embodiments of the present invention, the term heteroaryl, as used herein, refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl,oxadiazolyl, thiophenyl, furanyl, quinolinyl, isoquinolinyl, and the like.

It will be appreciated that aryl and heteroaryl groups can be unsubstituted or substituted, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

The term carboxylic acid as used herein refers to a group of formula —CO₂H.

The terms halo and halogen as used herein refer to an atom selected from fluorine, chlorine, bromine, and iodine.

The term heteroaliphatic, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term heterocyclic, as used herein, refers to an aromatic or non-aromatic, partially unsaturated or fully saturated, 3- to 10-membered ring system, which includes single rings of 3 to 8 atoms in size and bi- and tri-cyclic ring systems which may include aromatic five- or six-membered aryl or aromatic heterocyclic groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocyclic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring.

The term aromatic heterocyclic, as used herein, refers to a cyclic aromatic radical having from five to ten ring atoms of which one ring atom is selected from sulfur, oxygen, and nitrogen; zero, one, or two ring atoms are additional heteroatoms independently selected from sulfur, oxygen, and nitrogen; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like. Aromatic heterocyclic groups can be unsubstituted or substituted with substituents selected from the group consisting of branched and unbranched alkyl, alkenyl, alkynyl, haloalkyl, alkoxy, thioalkoxy, amino, alkylamino, dialkylamino, trialkylamino, acylamino, cyano, hydroxy, halo, mercapto, nitro, carboxyaldehyde, carboxy, alkoxycarbonyl, and carboxamide.

Specific heterocyclic and aromatic heterocyclic groups that may be included in the compounds of the invention include: 3-methyl-4-(3-methylphenyl)piperazine, 3 methylpiperidine, 4-(bis-(4-fluorophenyl)methyl)piperazine, 4-(diphenylmethyl)piperazine, 4-(ethoxycarbonyl)piperazine, 4-(ethoxycarbonylmethyl)piperazine, 4-(phenylmethyl)piperazine, 4-(1-phenylethyl)piperazine, 4-(1,1-dimethylethoxycarbonyl)piperazine, 4-(2-(bis-(2-propenyl) amino)ethyl)piperazine, 4-(2-(diethylamino)ethyl)piperazine, 4-(2-chlorophenyl)piperazine, 4-(2-cyanophenyl)piperazine, 4-(2-ethoxyphenyl)piperazine, 4-(2-ethylphenyl)piperazine, 4-(2-fluorophenyl)piperazine, 4-(2-hydroxyethyl)piperazine, 4-(2-methoxyethyl)piperazine, 4-(2-methoxyphenyl)piperazine, 4-(2-methylphenyl)piperazine, 4-(2-methylthiophenyl) piperazine, 4-(2-nitrophenyl)piperazine, 4-(2-nitrophenyl)piperazine, 4-(2-phenylethyl)piperazine, 4-(2-pyridyl)piperazine, 4-(2-pyrimidinyl)piperazine, 4-(2,3-dimethylphenyl)piperazine, 4-(2,4-difluorophenyl)piperazine, 4-(2,4-dimethoxyphenyl)piperazine, 4-(2,4-dimethylphenyl)piperazine, 4-(2,5-dimethylphenyl)piperazine, 4-(2,6-dimethylphenyl)piperazine, 4-(3-chlorophenyl)piperazine, 4-(3-methylphenyl)piperazine, 4-(3-trifluoromethylphenyl)piperazine, 4-(3,4-dichlorophenyl)piperazine, 4-3,4-dimethoxyphenyl)piperazine, 4-(3,4-dimethylphenyl)piperazine, 4-(3,4-methylenedioxyphenyl)piperazine, 4-(3,4,5-trimethoxyphenyl)piperazine, 4-(3,5-dichlorophenyl)piperazine, 4-(3,5-dimethoxyphenyl)piperazine, 4-(4-(phenylmethoxy)phenyl)piperazine, 4-(4-(3,1-dimethylethyl)phenylmethyl)piperazine, 4-(4-chloro-3-trifluoromethylphenyl)piperazine, 4-(4-chlorophenyl)-3-methylpiperazine, 4-(4-chlorophenyl)piperazine, 4-(4-chlorophenyl)piperazine, 4-(4-chlorophenylmethyl)piperazine, 4-(4-fluorophenyl)piperazine, 4-(4-methoxyphenyl)piperazine, 4-(4-methylphenyl)piperazine, 4-(4-nitrophenyl)piperazine, 4-(4-trifluoromethylphenyl)piperazine, 4-cyclohexylpiperazine, 4-ethylpiperazine, 4-hydroxy-4-(4-chlorophenyl)methylpiperidine, 4-hydroxy-4-phenylpiperidine, 4-hydroxypyrrolidine, 4-methylpiperazine, 4-phenylpiperazine, 4-piperidinylpiperazine, 4-(2-furanyl)carbonyl)piperazine, 4-((1,3-dioxolan-5-yl)methyl)piperazine, 6-fluoro-1,2,3,4-tetrahydro-2-methylquinoline, 1,4-diazacylcloheptane, 2,3-dihydroindolyl, 3,3-dimethylpiperidine, 4,4-ethylenedioxypiperidine, 1,2,3,4-tetrahydroisoquinoline, 1,2,3,4-tetrahydroquinoline, azacyclooctane, decahydroquinoline, piperazine, piperidine, pyrrolidine, thiomorpholine, and triazole.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)_{0 4}N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched)alkylene)O—N(R^{∘})₂; or —(C₁₋₄ straight or branched)alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{}, -(haloR^{}), —(CH₂)₀₋₂OH, —(CH₂)₀₋₂OR^{}, —(CH₂)_{0 2}CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)_{0 2}NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}or —SSR^{} wherein each R^{} is unsubstituted or where preceded by “halo” is substituted only with one or more halogens, and is independently selected from C₁₋₄ aliphatic, —CH₂Ph, —O(CH₂)_{0 1}Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{*} include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{\}, NR^{\}₂, —C(O)R^{\}, —C(O)OR^{\}, —C(O)C(O)R^{\}, —C(O)CH₂C(O)R^{\}, —S(O)₂R^{\}, —S(O)₂NR^{\}₂, —C(S)NR^{\}₂, —C(NH)NR^{\}₂, or —N(R^{\})S(O)₂R^{\}; wherein each R^{\} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{\}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{\} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term arylalkyl refers to alkyl groups in which a hydrogen atom has been replaced with an aryl group. Such groups include, without limitation, benzyl, cinnamyl, and dihyrocinnamyl.

The term heteroatom means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term unsaturated, as used herein, means that a moiety has one or more units of unsaturation.

As used herein, the term partially unsaturated refers to a ring moiety that includes at least one double or triple bond. The term “partially unsaturated” is intended to encompass rings having multiple sites of unsaturation, but is not intended to include aryl or heteroaryl moieties, as herein defined.

Additionally, unless otherwise stated, structures depicted herein are also meant to include compounds that differ only in the presence of one or more isotopically enriched positions of the compound. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

One of ordinary skill in the art will appreciate that the synthetic methods, as described herein, utilize a variety of protecting groups. By the term “protecting group,” as used herein, it is meant that a particular functional moiety, e.g., O, S, or N, is masked or blocked, permitting, if desired, a reaction to be carried out selectively at another reactive site in a multifunctional compound. Suitable protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. In certain embodiments, a protecting group reacts selectively in good yield to give a protected substrate that is stable to the projected reactions; the protecting group is preferably selectively removable by readily available, preferably non-toxic reagents that do not attack the other functional groups; the protecting group forms a separable derivative (more preferably without the generation of new stereogenic centers); and the protecting group will preferably have a minimum of additional functionality to avoid further sites of reaction. As detailed herein, oxygen, sulfur, nitrogen, and carbon protecting groups may be utilized. By way of non-limiting example, hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described by Greene and Wuts (supra).

As used herein, the phrase “natural amino acid side-chain group” refers to the side-chain group of any of the 20 amino acids naturally occuring in proteins.

As used herein, the phrase “unnatural amino acid side-chain group” refers to amino acids not included in the list of 20 amino acids naturally occuring in proteins, as described above. Such amino acids include the D-isomer of any of the 20 naturally occuring amino acids. Unnatural amino acids also include homoserine, ornithine, norleucine, and thyroxine. Other unnatural amino acids side-chains are well known to one of ordinary skill in the art and include unnatural aliphatic side chains. Other unnatural amino acids include modified amino acids, including those that are N-alkylated, cyclized, phosphorylated, acetylated, amidated, azidylated, labelled, and the like. In some embodiments, an unnatural amino acid is a D-isomer. In some embodiments, an unnatural amino acid is a L-isomer.

A compound of the present invention may be tethered to a detectable moiety. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry (infra).

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, CyS, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

As used herein and in the claims, the singular forms “a”, “an”, and “the” include the plural reference unless the context clearly indicates otherwise. Thus, for example, a reference to “a compound” includes a plurality of such compounds.

### DETAILED DESCRIPTION OF CERTAIN EMBODIMENTS OF THE INVENTION

The present invention provides fluorinated phospholipid compounds and methods of using the compounds to deliver agents to cells. The fluorinated phospholipid compositions described herein allow one to deliver agents such as macromolecules and small molecules (e.g., membrane-impermeable small molecules, small molecules associated with macromolecules (e.g., biotin associated with avidin or streptavidin)) into living cells. These compositions have a wide rage of applications, including, e.g., therapeutic, diagnostic, and in vitro applications. Exemplary agents for delivery include, but are not limited to, nucleic acids (e.g. DNA or RNA, such as siRNAs, shRNAs, tRNAs, and ribozymes), peptides (including multimeric proteins, protein complexes, antibodies, etc.), lipids, carbohydrates, hormones, small molecules, etc., and/or combinations thereof.

The present disclosure provides description of fluorinated phospholipids that are non-toxic and mediate highly efficient delivery of agents into living cells. In certain embodiments, the provided fluorinated phospolipid compounds have increased hydrophobicity and affinity for membranes relative to non-fluorinated lipids. In some embodiments, the fluorinated phospholipid compounds can enter cells by mechanisms that include endocytosis. In some embodiments, fluorinated phospholipid compounds described herein have properties that deliver agents to the cytoplasm and/or membranes of living cells, while avoiding or minimizing delivery of the agents into cell nuclei. In some embodiments, provided fluorinated phospholipid compounds have properties that allow a therapeutic agent to be delivered into the cell's cytoplasm, exclusive of the nucleus. In some embodiments, fluorinated phospholipid compounds described herein are non-cationic and may deliver an agent while causing diminished or no cytotoxicity (such as, for example, that associated with disruption of cell membranes).

### Fluorinated Phospholipid Compounds

In certain embodiments, provided fluorinated phospholipid compounds comprise a non-cationic phospholipid, a linker, and an agent for delivery to a cell, wherein at least one of hydrocarbon chain of the phospolipid is fluorinated, and wherein the phospholipid, the linker, and the agent are covalently linked. In some embodiments, provided fluorinated phospholipid compounds are of the formula:

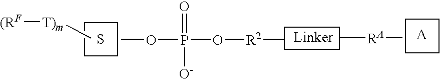

wherein:

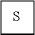

is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{A} is a covalent bond or an optionally substituted moiety derived from conjugating an optionally substituted thiol-reactive, amine-reactive, or hydroxyl-reactive moiety with a thiol, amine, or hydroxyl group of the agent;

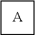

is a therapeutic agent;
each occurrence of n is an integer from 0 to 30, inclusive, wherein at least one occurrence of n is non-zero; and
m is an integer from 1 to 2, inclusive, wherein m is 1 when

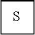

is a covalent bond.

In certain embodiments,

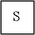

is a covalent bond. In some embodiments,

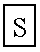

is optionally substituted acyl. In some embodiments,

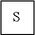

is optionally substituted heteroaliphatic. In some embodiments,

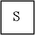

is optionally substituted heteroaryl. In some embodiments,

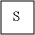

is optionally substituted heterocyclic.

In some embodiments,

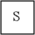

is optionally substituted aliphatic. In certain embodiments,

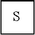

is an optionally substituted C₁₋₂₄ aliphatic group. In certain embodiments,

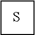

is an optionally substituted C₁₋₁₂ aliphatic group. In certain embodiments,

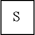

is an optionally substituted C₁₋₈ aliphatic group. In certain embodiments,

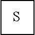

is

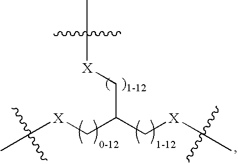

wherein X is N, O, or S. In certain embodiments,

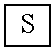

is

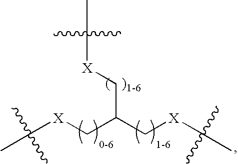

wherein X is N, O, or S. In some embodiments, X is O.

One of ordinary skill in the art will appreciate that polyol compounds are useful when preparing compound of the present invention. In certain embodiments, a diol or triol can be used in accordance with the present invention, and the present disclosure contemplates any

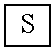

derived from a diol or triol.

In some embodiments,

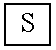

is

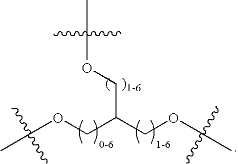

In some embodiments,

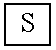

is

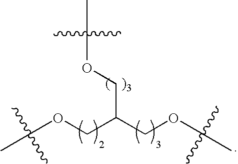

In some embodiments,

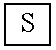

is

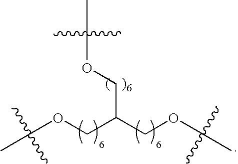

In some embodiments,

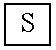

is

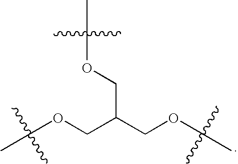

In some embodiments,

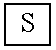

is

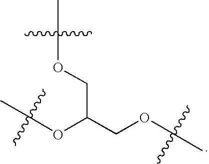

In some embodiments,

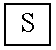

is optionally substituted aryl. In some embodiments,

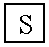

is an optionally substituted 6-membered aryl group. In some embodiments,

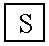

is

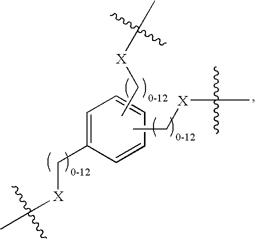

wherein X is N, O, or S. In some embodiments,

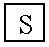

is

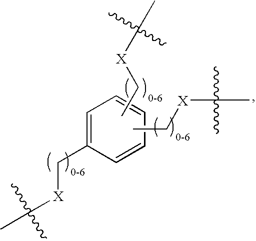

wherein X is N, O, or S. In some embodiments,

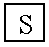

is

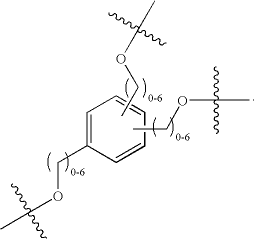

In some embodiments,

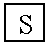

is

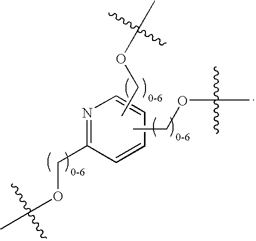

In certain embodiments,

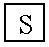

is

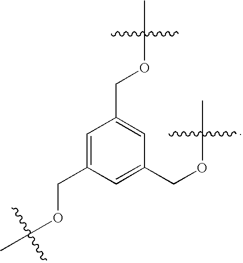

In certain embodiments,

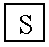

is

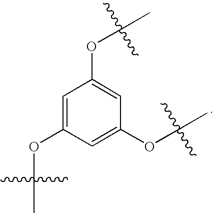

In some embodiments, T is a covalent bond. In certain embodiments, each occurrence of T is independently a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—. In some embodiments, T is a bivalent C₁₋₃₀ hydrocarbon chain wherein one or more methylene units of T are optionally and independently replaced as described above. In some embodiments, T is a bivalent C₁₋₂₀ hydrocarbon chain wherein one or more methylene units of T are optionally and independently replaced as described above. In some embodiments, T is a bivalent C₁₋₁₂ hydrocarbon chain wherein one or more methylene units of T are optionally and independently replaced as described above.

In certain embodiments, one, two, or three methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—. In some embodiments, one or two methylene units of T are replaced by —O— or —C(O)—. In some embodiments, each occurrence of T is independently a bivalent C₁₋₃₀ hydrocarbon chain wherein one or two methylene units of T are optionally and independently replaced by —CF₂—, —O—, —S—, —N(R)—, —C(O)—, —C(O)O—, —OC(O)—, —N(R)C(O)—, or —C(O)N(R)—. In some embodiments, T is a moiety selected from the group consisting of —C_{n′}H_{(2n′)}C(O)—, —C_{n′}H_{(2n′)}OC(O)—, and —C_{n′}H_{(2n′)}N(R)C(O)—; wherein n′ is an integer from 1 to 28, inclusive. In some embodiments, T is —C_{n′}H_{(2n′)}C(O)—. In some embodiments, n′ is an integer from 1 to 10, inclusive.

In some embodiments, m is 1. In some embodiments, m is 2.

As described above, each occurrence of R^{F} is a group having the formula —CₙF₍₂ₙ₊₁₎. In certain embodiments, n is 0 and R^{F} is absent. In some embodiments, n is an integer from 1 to 30, inclusive. In some embodiments, n is an integer from 1 to 20, inclusive. In some embodiments, n is an integer from 1 to 10, inclusive. In some embodiments, n is an integer from 1 to 6, inclusive. In some embodiments, n is 6. In some embodiments, n is 4. In some embodiments, n is 1.

In some embodiments, R² is a covalent bond. In some embodiments, R² is an optionally substituted bivalent, straight or branched, saturated or unsaturated, C₁₋₂₀ aliphatic or C₁₋₂₀ heteroaliphatic chain. In some embodiments, one or two methylene units of R² are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur. In some embodiments, one or two methylene units of R² are optionally and independently replaced by a 5-10 membered heteroaryl having 1-4 heteroatoms independently selected from nitrogen, oxygen, or sulfur. In some embodiments, one methylene unit of R² is replaced by a triazole moiety. In other embodiments, R² comprises an amino acid residue.

In certain embodiments, R² is —(C₁₋₁₂ aliphatic)-NH—. In certain embodiments, R² is —C₂H₄—NH—. In certain embodiments, R² is

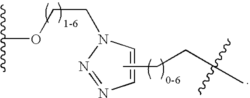

In certain embodiments, R² is

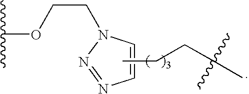

In certain embodiments, R² is

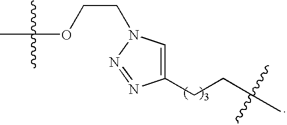

In other embodiments, R² is

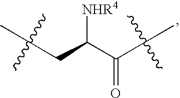

wherein R⁴ is hydrogen or a protecting group. One of ordinary skill will appreciate that a variety of protecting groups may be used, including those described above. All natural or unnatural amino acid side chains are contemplated and within the scope of the invention. In some embodiments, an amino acid side chain is non-cationic. In some embodiments, R⁴ is hydrogen. In other embodiments, R⁴ is acyl.

In some embodiments, R^{A} is a covalent bond. In other embodiments, R^{A} is an optionally substituted moiety derived from a cross linking agent capable of conjugating a heteroatom of the linker with a thiol or amine of the agent. In some embodiments, R^{A} is an optionally substituted moiety derived from a cross linking agent capable of conjugating a amine or hydroxyl of the linker with a thiol or amine of the agent. Suitable crosslinkers from which R^{A} may be derived are widely known in the art (see, for example, Pierce Technical Handbook: infra), including bromoacetic NHS ester, 6-(iodoacetamido)caproic acid NHS ester, maleimidoacetic acid NHS ester, maleimidobenzoic acid NHS ester, to name but a few. In certain embodiments, a crosslinker is MBS (m-maleimidobenzoyl acid N-Hydroxysuccinimidyl ester). In some embodiments, R^{A} is derived from a cross linking agent comprising a NHS ester. In some embodiments, R^{A} is derived from a cross linking agent comprising a maleimide. In some embodiments, R^{A} is derived from a cross linking agent comprising a maleimide and a NHS ester. In some embodiments, the cross linking agent further comprises a PEG moiety.

In some embodiments, the linkage between R^{A} and

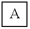

comprises a disulfide bond. In some embodiments, the bond between R^{A} and

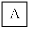

is a disulfide bond. In some embodiments, cleavage of a disulfide bond between R^{A} and

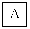

is facilitated by endogenous glutathione. While not wishing to be bound by any particular theory, it is believed that higher concentrations of glutathione present in tumor cells may be useful for promoting the selective or preferential cleavage of chemotherapeutics from fluorinated phospholipid compounds in tumor cells relative to non-tumor cells.

In some embodiments, R^{A} is —CH₂—. In some embodiments, R^{A} is —C(O)—. In other embodiments, R^{A} is —CH₂C(O)—. In some embodiments, R^{A} is

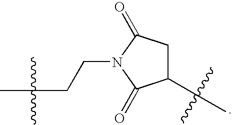

In some embodiments, R^{A} is

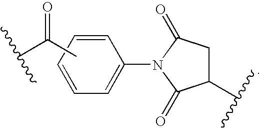

One of ordinary skill in the art will appreciate that R^{A} groups, depending on the cross linking agent used, may contain a thioester bond. In some embodiments, R^{A} may contain a thioether bond.

In certain embodiments, R^{A} is an optionally substituted moiety derived from conjugating an optionally substituted thiol-reactive, amine-reactive, or hydroxyl-reactive moiety with a thiol, amine, or hydroxyl group of the agent.

In some embodiments, crosslinking may be accomplished using click chemistry. In certain embodiments, an alkyne moiety present on an agent is conjugated with an azide moiety on a linker to provide a triazole moiety. In other embodiments, an alkyne moiety present on a linker is conjugated with an alkyne moiety on an agent to provide a triazole moiety. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57.

In some embodiments, provided fluorinated phospholipid compositions comprise a non-cationic phospholipid, a linker, and an agent for delivery to a cell, wherein at least one of hydrocarbon chain of the phospolipid is fluorinated, and wherein the phospholipid and the agent are not covalently linked.

In some embodiments, provided fluorinated phospholipid compounds are of the formula:

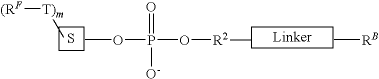

wherein:

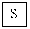

is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{B} is an optionally substituted moiety capable of forming a non-covalent interaction with a therapeutic agent;
each occurrence of n is an integer from 0 to 30, inclusive, wherein at least one occurrence of n is non-zero; and
m is an integer from 1 to 2, inclusive, wherein m is 1 when

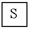

is a covalent bond.

In some embodiments, R^{B} is streptavidin. In some embodiments, R^{B} is biotin. In some embodiments, R^{B} is beta-cyclodextrin. In some embodiments, R^{B} is alpha-cyclodextrin. In some embodiments, R^{B} is an antigen. In some embodiments, R^{B} is an antigen present on tumor cells.

One of ordinary skill in the art will be familiar with techniques of forming non-covalent interactions with a therapeutic agent. Depending upon the selection of R^{B}, a therapeutic agent may form a non-covalent interaction with R^{B}. The agent may be modified with a suitable moiety in order to bring about the desired non-covalent interaction. Examples of such interactions, by way of non-limiting example, include streptavidin with biotin, beta-cyclodextrin with small hydrophobic compounds, alpha-cyclodextrin with small hydrophobic compounds, organometallic complexes, proteins with small molecules, and antigens with antibodies.

In certain embodiments, provided compositions are of the formula:

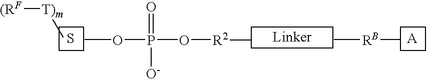

wherein each of R^{F}, T, m,

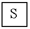

### R², R^{B},

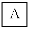

and the linker is as defined above and described in classes and subclasses herein; wherein R^{B} and

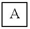

are not covalently linked.

In certain embodiments, the present invention provides a method comprising the steps of:
a) providing a non-cationic fluorinated phospholipid of the formula:

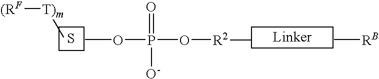

wherein:

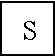

is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —C_{F}F₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{B} is an optionally substituted moiety capable of forming a non-covalent interaction with a therapeutic agent;
each occurrence of n is an integer from 0 to 30, inclusive, wherein at least one occurrence of n is non-zero; and
m is an integer from 1 to 2, inclusive, wherein m is 1 when

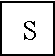

is a covalent bond; and
b) contacting the fluorinated phospholipid with an agent for delivery to a cell to form a compound of formula:

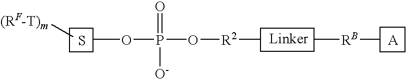

wherein R^{B} and

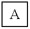

are not covalently linked.

In some embodiments, the present invention provides a composition comprising:

a) a non-cationic phospholipid, wherein at least one hydrocarbon chain of the phospolipid is fluorinated and the compound is of the formula:

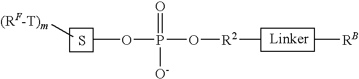

wherein:

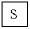

is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{B} is an optionally substituted moiety capable of forming a non-covalent interaction with a therapeutic agent;
each occurrence of n is an integer from 0 to 30, inclusive, wherein at least one occurrence of n is non-zero; and
m is an integer from 1 to 2, inclusive, wherein m is 1 when

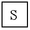

is a covalent bond; and

b) a therapeutic agent

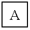

non-covalently linked to R^{B}.

In certain embodiments, provided non-cationic fluorinated phospholipids are of the formula:

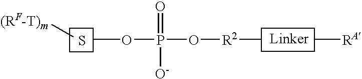

wherein:

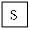

is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{A′} is hydrogen or an optionally substituted thiol-reactive, amine-reactive, or hydroxyl-reactive moiety;
each occurrence of n is an integer from 0 to 30, inclusive, wherein at least one occurrence of n is non-zero; and
m is an integer from 1 to 2, inclusive, wherein m is 1 when

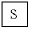

is a covalent bond.

In certain embodiments, R^{A′} is hydrogen. In certain embodiments, R^{A′} is an optionally substituted thiol-reactive moiety. In certain embodiments, R^{A′} is an optionally substituted amine-reactive moiety. In certain embodiments, R^{A′} is an optionally substituted hydroxyl-reactive moiety.

In some embodiments, R^{A′} is selected from the group consisting of maleimides, esters, alkyl halides, iodoacetamides, and thiols. In certain embodiments, R^{A′} is an NHS ester. In some embodiments, R^{A′} is an optionally substituted moiety capable of conjugation with a with a thiol, amine, or hydroxyl group of the agent. Suitable moieties from which R^{A′} may be derived are widely known in the art (see, for example, Pierce Technical Handbook: infra), including bromoacetic NHS ester, 6-(iodoacetamido)caproic acid NHS ester, maleimidoacetic acid NHS ester, maleimidobenzoic acid NHS ester, to name but a few. In certain embodiments, R^{A′} is MBS (m-maleimidobenzoyl acid N-Hydroxysuccinimidyl ester).

In certain embodiments, R^{A′} is other than an NHS ester. In certain embodiments, R^{A′} is other than a carboxylic acid.

In some embodiments, a fluorinated phospholipid compound is provided as vesicles (i.e., structures characterized by the presence of one or more membranes which form one or more internal voids). In some embodiments, a composition of a fluorinated phospholipid compound includes a stabilizing agent (e.g., a lipid, a surfactant, a peptide, a polymeric material, or a combination thereof.

Methods of conjugating thiol-reactive, amine-reactive, or hydroxyl-reactive moieties to thiols, amines, or hydroxyl groups are known in the art and include those described in WO/2007/113531, and the Pierce Technical Handbook (infra).

In certain embodiments, the present invention provides a method of making a compound, the method comprising contacting an agent for delivery to a cell with a phospholipid covalently linked to a linker, wherein at least one hydrocarbon chain of the phospholipid is fluorinated, wherein the phospholipid, and the linker are covalently linked, and wherein the linker comprises a moiety that is reactive with a moiety present on the agent.

In certain embodiments, the method comprises:
a) providing a non-cationic fluorinated phospholipid of the formula:

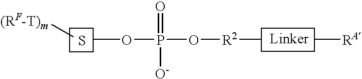

wherein:

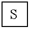

is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{A′} is hydrogen or an optionally substituted thiol-reactive, amine-reactive, or hydroxyl-reactive moiety;
each occurrence of n is an integer from 0 to 30, inclusive, wherein at least one occurrence of n is non-zero; and
m is an integer from 1 to 2, inclusive, wherein m is 1 when

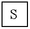

is a covalent bond; and
b) contacting the fluorinated phospholipid with an agent for delivery to a cell to form a compound of formula:

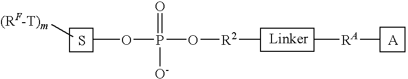

wherein:
R^{A} is a covalent bond or an optionally substituted moiety derived from conjugating an optionally substituted thiol-reactive, amine-reactive, or hydroxyl-reactive moiety with a thiol, amine, or hydroxyl group of the agent; and

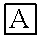

is a therapeutic agent.

### Linkers

A variety of linkers can be used to produce fluorinated lipid compounds. In some embodiments, the linker comprises a peptide. In some embodiments, the linker is a peptide comprising between 1 and 40 amino acid residues, wherein each residue may have a natural or unnatural side chain group. In some embodiments, the linker is a peptide comprising between 1 and 25 amino acid residues, wherein each residue may have a natural or unnatural side chain group. In some embodiments, the linker is a peptide comprising between 1 and 10 amino acid residues, wherein each residue may have a natural or unnatural side chain group. In some embodiments, the peptide has 3 to 10 amino acid residues. In certain embodiments, the linker includes polyglycine.

In some embodiments, a linker comprises polyethylene glycol, or a related polymer (e.g., polypropyleneglycol, polymethacrylamide, polydimethacrylamide, polyhydroxyethylacrylate, polyhydroxypropylmethacrylate, polyoxyalkene). In some embodiments, a linker is other than PEG.

In some embodiments, a linker is an optionally substituted bivalent acyl moiety. In some embodiments, a linker is an optionally substituted bivalent aliphatic moiety. In some embodiments, a linker is an optionally substituted bivalent heteroaliphatic moiety. In some embodiments, a linker is an optionally substituted bivalent aryl moiety. In some embodiments, a linker is an optionally substituted bivalent heteroaryl moiety. In some embodiments, a linker is an optionally substituted bivalent heterocyclic moiety.

In certain embodiments, a linker is an optionally substituted, straight or branched, bivalent C₁₋₂₀ aliphatic group wherein one or more methylene units are optionally and independently replaced by —O—, —S—, —N(R)—, —C(O)—, —C(O)O—, —OC(O)—, —N(R)C(O)—, or —C(O)N(R)—.

Linkers that have various types of functional groups are suitable for producing the fluorinated lipid compounds described herein. In some embodiments, a linker is linked to an agent via a thiol-reactive moiety (e.g., a maleimide, a pyridyldisulfide, an iodoacetimide). In some embodiments, a linker is linked via an amine-reactive moiety (e.g., a carbodiimide, a succinimidyl ester). In some embodiments, a linker is linked to an agent via a moiety derived from a reagent capable of conjugating a heteroatom of the linker with a thiol, amine, or hydroxyl group of the agent.

Amine-carboxylic acid and thiol-carboxylic acid cross-linking, maleimide-sulfhydryl coupling chemistries (e.g., the maleimidobenzoyl-N-hydroxysuccinimide ester (MBS) method), etc., may be used. Peptides can conveniently be attached to linkers via amine or thiol groups in lysine or cysteine side chains respectively, or by an N-terminal amino group. Nucleic acids such as RNAs can be synthesized with a terminal amino group.

In certain embodiments, an agent is attached to a fluorinated phospholipid via a cleavable linkage so that the agent is released from the fluorinated lipid following intracellular delivery. The cleavage of the linker may be via chemical cleavage, acid cleavage, beta-glucosidase-mediated cleavage, calpain-mediated cleavage, aminopeptidase-mediated cleavage, protease-mediated cleavage, lipase-mediated cleavage, or light-directed cleavage, to name but a few. Cleavable linkages include disulfide bonds, acid-labile thioesters, peptide bonds, acetal bonds, ketal bonds, aminal bonds, hemiaminal ethers, etc. (Oishi et al., 2005, J. Am. Chem. Soc., 127:1624; Biochem. Biophys. Res. Commun., 1981, 102, 1048-1054; Bioconjugate Chem. 2007, 18, 293-296; Bioconjugate Chem., 2004, 15, 1254-1263; J. Med. Chem. 1985, 28, 51-57; J. Biol. Chem., 2005, 280, 40632-40641; J. Org. Chem. 1990, 55, 5867-5877; J. Org. Chem. 1997, 62, 1363-1367; J. Med. Chem. 2000, 43, 475-487; Macromolecules, 2005, 38, 10757; FEBS Letters, 1979, 98, 119-122; J. Med. Chem, 1980, 23, 1166-1170; J. Med. Chem, 1980, 23, 1171-1174; each of which is incorporated herein by reference).

Any linker that contains or forms such a bond could be employed. In some embodiments, the linker contains a peptide sequence that includes a cleavage site for an intracellular enzyme. In some embodiments, the linker contains a peptide sequence that includes a cleavage site for an intracellular protease. In some embodiments, the linker includes a moiety sensitive to cleavage by an intracellular lipase. In some embodiments, the linker includes a moiety sensitive to cleavage by an intracellular aminopeptidase. In some embodiments, the linker contains a peptide sequence that includes a cleavage site for an intracellular beta-glucosidase. In some embodiments, the linker contains a peptide sequence that includes a cleavage site for an intracellular carboxyesterase.

In some embodiments, a linker is a biodegradable linker. In some embodiments, a linker is enzyme sensitive. In some embodiments, a linker is cleavable under acidic conditions. In some embodiments, a linker is cleavable in the acidic environment of an endosome. In some embodiments, a linker is cleavable in the acidic environment of a lysosome. In some embodiments, a linker is selected such that the relative acidic environment of a tumor cell facilitates linker cleavage. In some embodiments, a linker has the structure:

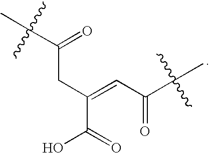

In some embodiments, a linker has the structure:

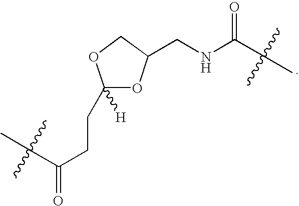

In some embodiments, a linker has the structure:

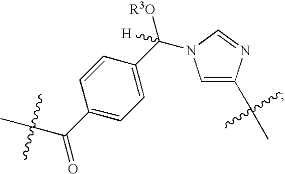

wherein R³ is C₁₋₆ aliphatic. In certain embodiments, R³ is ethyl.

In some embodiments, a linker is beta-glucosidase sensitive. In some embodiments, a linker has the structure:

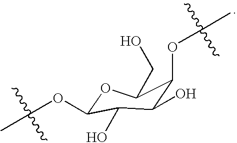

In some embodiments, a linker is calpain sensitive. In some embodiments, a linker has the structure:

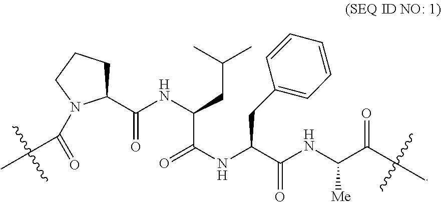

In some embodiments, a linker is carboxyesterase sensitive. In some embodiments, a linker has the structure:

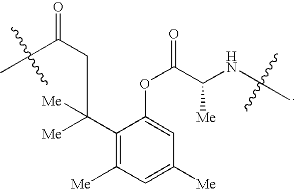

In some embodiments, a linker has the structure:

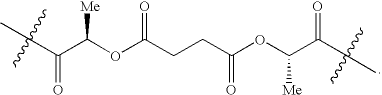

In some embodiments, a linker is lysosomal aminopeptidase sensitive. In some embodiments, a linker has the structure:

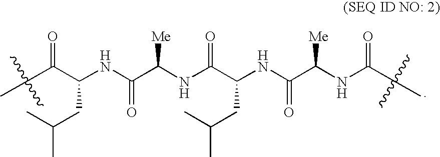

General information on conjugation methods, reactive moieties, and cross-linking is found, e.g., in the journal Bioconjugate Chemistry, published by the American Chemical Society, Columbus Ohio, PO Box 3337, Columbus, Ohio, 43210;“Cross-Linking,” Pierce Chemical Technical Library, available at the Pierce web site and originally published in the 1994-95 Pierce Catalog, and references cited therein; Wong S S, Chemistry of Protein Conjugation and Cross-linking, CRC Press Publishers, Boca Raton, 1991; and Hermanson, G. T., Bioconjugate Techniques, Academic Press, Inc., San Diego, 1996.

It is to be understood that the compositions in accordance with the invention can be made in any suitable manner, and the invention is in no way limited to compositions that can be produced using the methods described herein. Selection of an appropriate method may require attention to the properties of the particular moieties being linked.

If desired, various methods may be used to separate fluorinated phospholipids with an attached linker and agent, from lipids to which the linker or agent has not become attached, or to separate lipids having different numbers of agents attached thereto. For example, size exclusion chromatography or agarose gel electrophoresis can be used to separate populations of fluorinated phospholipids having different numbers of agents attached thereto and/or to separate phospholipids from other molecules. Some methods include size-exclusion or anion-exchange chromatography.

In certain embodiments, provided compounds are of the formula:

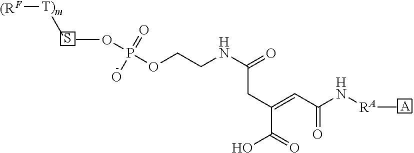

wherein each of R^{F}, T, m,

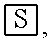

### R^{A}, and

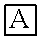

is as defined above and described in classes and subclasses herein.

In certain embodiments, provided compounds are of the formula:

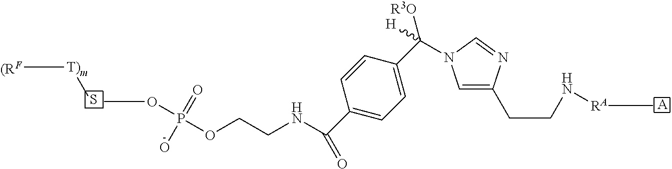

wherein each of R³, R^{F}, T, m,

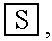

### R^{A}, and

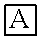

is as defined above and described in classes and subclasses herein.

In certain embodiments, provided compounds are of the formula:

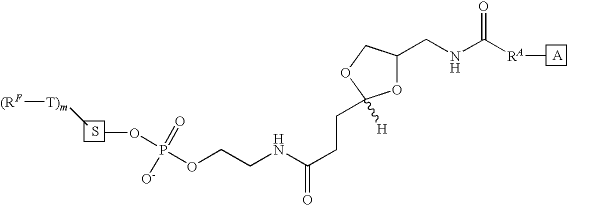

wherein each of R^{F}, T, m,

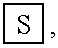

### R^{A}, and

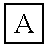

is as defined above and described in classes and subclasses herein.

In certain embodiments, provided compounds are of the formula:

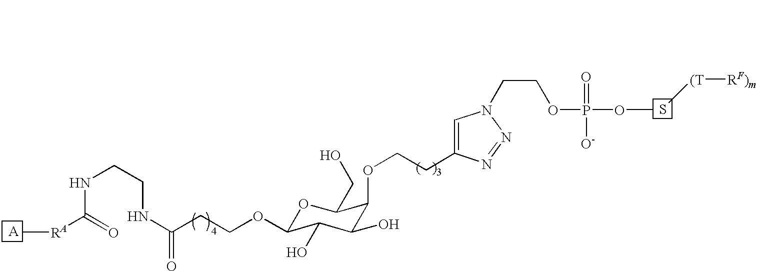

wherein each of R^{F}, T, m,

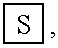

### R^{A}, and

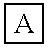

is as defined above and described in classes and subclasses herein.

In certain embodiments, provided compounds are of the formula:

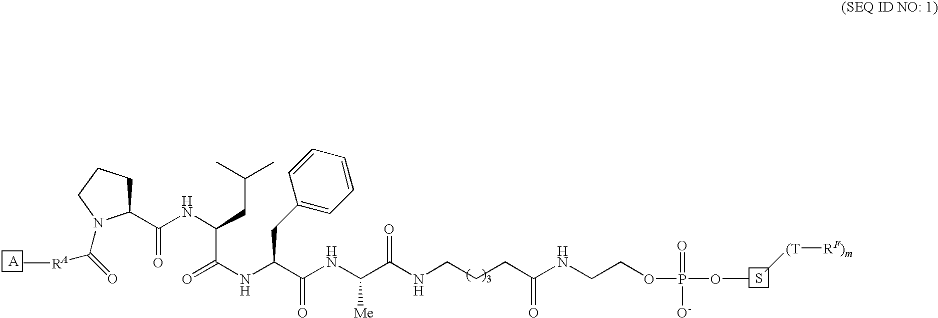

wherein each of R^{F}, T, m,

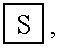

### R^{A}, and

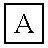

is as defined above and described in classes and subclasses herein.

In certain embodiments, provided compounds are of the formula:

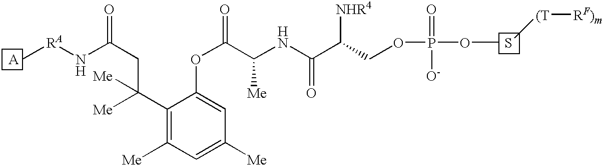

wherein each of R⁴, R^{F}, T, m,

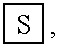

### R^{A}, and

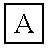

is as defined above and described in classes and subclasses herein.

In certain embodiments, provided compounds are of the formula:

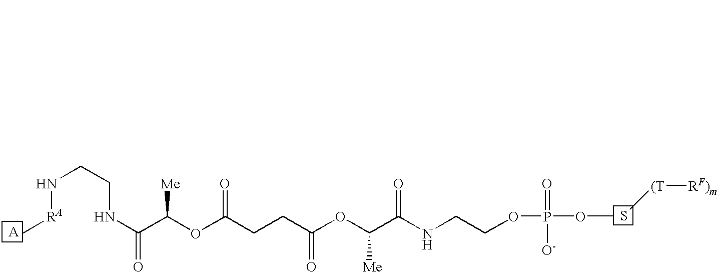

wherein each of R^{F}, T, m,

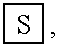

### R^{A}, and

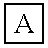

is as defined above and described in classes and subclasses herein.

In certain embodiments, provided compounds are of the formula:

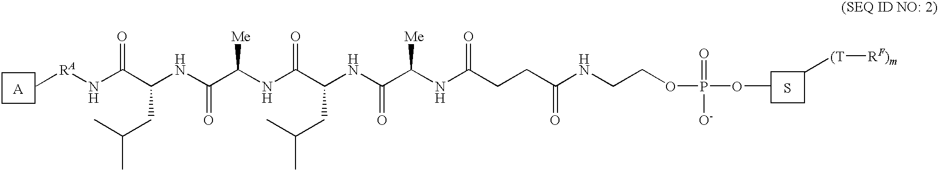

wherein each of R^{F}, T, m,

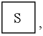

### R^{A}, and

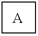

is as defined above and described in classes and subclasses herein.

In certain embodiments, provided compounds are of the formula:

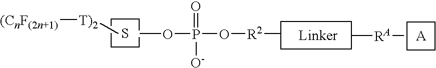

wherein each of T, n,

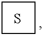

### R², R^{A},

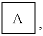

and the linker is as defined above and described in classes and subclasses herein.

In certain embodiments, provided compounds are of the formula:

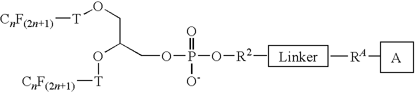

wherein each of T, n, R², R^{A},

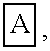

and the linker is as defined above and described in classes and subclasses herein.

In certain embodiments, provided compounds are of the formula:

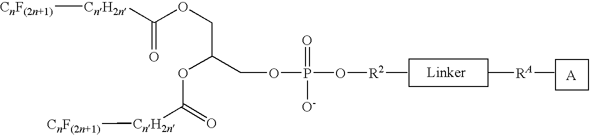

wherein each of n′, n, R², R^{A},

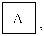

and the linker is as defined above and described in classes and subclasses herein.

In certain embodiments, provided compounds are of the formula:

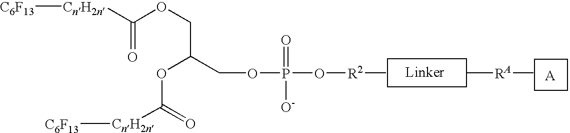

wherein each of n′, R², R^{A},

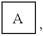

and the linker is as defined above and described in classes and subclasses herein.

In certain embodiments. provided compounds are of the formula:

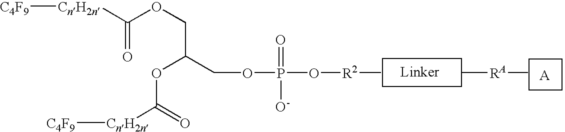

wherein each of n′, R², R^{A},

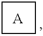

and the linker is as defined above and described in classes and subclasses herein.

In certain embodiments, provided compounds are of the formula:

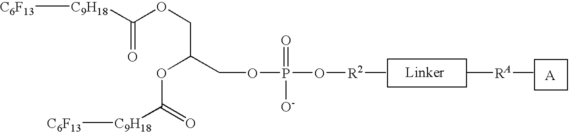

wherein each of R², R^{A},

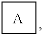

and the linker is as defined above and described in classes and subclasses herein.

While phosphate groups on compounds of the invention are typically shown without a proton, it will be appreciated that the present invention encompasses both protonated and deprotonated phosphate groups.

### Agents for Delivery

### Nucleic Acids

In certain embodiments, a fluorinated lipid is conjugated to a nucleic acid. Lipid-nucleic acid conjugate compounds can be used, e.g., to introduce nucleic acids encoding peptides, or to introduce nucleic acids that inhibit gene expression.

In certain embodiments, the nucleic acid is a DNA (e.g., an oligonucleotide, e.g., a DNA encoding a peptide). In certain embodiments, the DNA is a DNA comprising a peptide coding sequence and sequences that mediate and/or regulate expression of the peptide (e.g., a promoter sequence, a regulatory sequence, and the like).

In certain embodiments, a fluorinated lipid is conjugated to an RNA. In some embodiments, the RNA is an RNA that encodes a peptide. In some embodiments, the RNA is a functional RNA (i.e., the RNA molecule itself mediates biological functions, such as inhibition of gene expression). Examples of functional RNAs include RNAs that mediate RNAi and ribozymes.

RNAi is a process in which presence of an at least partly double stranded RNA molecule (dsRNA) in a eukaryotic cell leads to sequence-specific inhibition of gene expression. RNAi is described, e.g., in PCT Publication WO 01/75164; U.S. Patent Publications 2002/0086356 and 2003/0108923; Zamore et al., 2000, Cell, 101:25; and Elbashir et al., 2001, Genes Dev., 15:188; all of which are incorporated herein by reference.

Short dsRNAs having structures such as this, referred to as siRNAs, silence expression of genes that include a region that is substantially complementary to one of the two strands. This strand is referred to as the “antisense” or “guide” strand, with the other strand often being referred to as the “sense” strand. The siRNA is incorporated into a ribonucleoprotein complex termed the RNA-induced silencing complex (RISC) that contains member(s) of the Argonaute protein family. Following association of the siRNA with RISC, a helicase activity unwinds the duplex, allowing an alternative duplex to form the guide strand and a target mRNA containing a portion substantially complementary to the guide strand. An endonuclease activity associated with the Argonaute protein(s) present in RISC is responsible for “slicing” the target mRNA, which is then further degraded by cellular machinery.

Exogenous introduction of siRNAs into mammalian cells can effectively reduce the expression of target genes in a sequence-specific manner via the mechanism described above. A typical siRNA structure includes a 19 nucleotide double-stranded portion, comprising a guide strand and an antisense strand. Each strand has a 2 nt 3′ overhang. Typically the guide strand of the siRNA is perfectly complementary to its target gene and mRNA transcript over at least 17-19 contiguous nucleotides, and typically the two strands of the siRNA are perfectly complementary to each other over the duplex portion. However, as will be appreciated by one of ordinary skill in the art, perfect complementarity is not required. Instead, one or more mismatches in the duplex formed by the guide strand and the target mRNA is often tolerated, particularly at certain positions, without reducing the silencing activity below useful levels. For example, there may be 1, 2, 3, or even more mismatches between the target mRNA and the guide strand (disregarding the overhangs). Thus, as used herein, two nucleic acid portions such as a guide strand (disregarding overhangs) and a portion of a target mRNA that are “substantially complementary” may be perfectly complementary (i.e., they hybridize to one another to form a duplex in which each nucleotide is a member of a complementary base pair) or they may have a lesser degree of complementarity sufficient for hybridization to occur. One of ordinary skill in the art will appreciate that the two strands of the siRNA duplex need not be perfectly complementary. Typically at least 80%, at least 90%, or more of the nucleotides in the guide strand of an effective siRNA are complementary to the target mRNA over at least about 19 contiguous nucleotides. The effect of mismatches on silencing efficacy and the locations at which mismatches may most readily be tolerated are areas of active study (see, e.g. Reynolds et al., 2004, Nat. Biotechnol., 22:326; incorporated herein by reference).

Molecules having the appropriate structure and degree of complementarity to a target gene will exhibit a range of different silencing efficiencies. A variety of additional design criteria have been developed to assist in the selection of effective siRNA sequences. Numerous software programs that can be used to choose siRNA sequences that are predicted to be particularly effective to silence a target gene of choice are available (see, e.g., Yuan et al., 2004, Nuc. Acid. Res., 32:W130; and Santoyo et al., 2005, Bioinformatics, 21:1376; both of which are incorporated herein by reference).

RNAi may be effectively mediated by RNA molecules having a variety of structures that differ in one or more respects from that described above. For example, the length of the duplex can be varied (e.g., from about 17-29 nucleotides); the overhangs need not be present and, if present, their length and the identity of the nucleotides in the overhangs can vary (though most commonly symmetric dTdT overhangs are employed in synthetic siRNAs).

Short hairpin RNAs (shRNAs) is another class of RNAs capable of mediating RNA interference. An shRNA is a single RNA strand that contains two complementary regions that hybridize to one another to form a double-stranded “stem,” with the two complementary regions being connected by a single-stranded loop. shRNAs are processed intracellularly by Dicer to form an siRNA structure containing a guide strand and an antisense strand. In some embodiments, shRNAs are delivered exogenously to cells. In other embodiments, intracellular synthesis of shRNA is achieved by introducing a plasmid or vector containing a promoter operably linked to a template for transcription of the shRNA into the cell, e.g., to create a stable cell line or transgenic organism.

Sequence-specific cleavage of target mRNA is a widely used means of achieving gene silencing by exogenous delivery of short RNAi agents to cells. Additional mechanisms of sequence-specific silencing mediated by short RNA species are also known. For example, post-transcriptional gene silencing mediated by small RNA molecules can occur by mechanisms involving translational repression. Certain endogenously expressed RNA molecules form hairpin structures containing an imperfect duplex portion in which the duplex is interrupted by one or more mismatches and/or bulges. These hairpin structures are processed intracellularly to yield single-stranded RNA species referred to as known as microRNAs (miRNAs), which mediate translational repression of a target transcript to which they hybridize with less than perfect complementarity. siRNA-like molecules designed to mimic the structure of miRNA precursors have been shown to result in translational repression of target genes when administered to mammalian cells.

RNAi mechanisms and the structure of various RNA molecules known to mediate RNAi, e.g. siRNA, shRNA, miRNA and their precursors, have been extensively reviewed (see, e.g. Dykxhhorn et al., 2003, Nat. Rev. Mol. Cell. Biol., 4:457; Hannon and Rossi, 2004, Nature, 431:3761; and Meister and Tuschl, 2004, Nature, 431:343; all of which are incorporated herein by reference). It is to be expected that future developments will reveal additional mechanisms by which RNAi may be achieved and will reveal additional effective short RNAi agents. Any currently known or subsequently discovered short RNAi agents are within the scope of the present invention.

An RNAi agent that is conjugated to a fluorinated lipid in accordance with the present invention and/or is present in a composition in accordance with the invention may be designed to silence any eukaryotic gene. The gene can be a mammalian gene, e.g., a human gene. The gene can be a wild type gene, a mutant gene, an allele of a polymorphic gene, etc. The gene can be disease-associated, e.g., a gene whose over-expression, under-expression, or mutation is associated with or contributes to development or progression of a disease. For example, the gene can be oncogene.

Another class of functional RNAs is tRNAs. The structure and role of tRNAs in protein synthesis is well known (Soll and Rajbhandary, (eds.) tRNA: Structure, Biosynthesis, and Function, ASM Press, 1995). The cloverleaf shape of tRNAs includes several double-stranded “stems” that arise as a result of formation of intramolecular base pairs between complementary regions of the single tRNA strand. There is considerable interest in the synthesis of polypeptides that incorporate unnatural amino acids such as amino acid analogs or labeled amino acids at particular positions within the polypeptide chain (see, e.g., Kohrer and RajBhandary, “Proteins carrying one or more unnatural amino acids,” Chapter 33, In Ibba et al., (eds.), Aminoacyl-tRNA Synthetases, Landes Bioscience, 2004). One approach to synthesizing such polypeptides is to deliver a suppressor tRNA that is aminoacylated with an unnatural amino acid to a cell that expresses an mRNA that encodes the desired polypeptide but includes a nonsense codon at one or more positions. The nonsense codon is recognized by the suppressor tRNA, resulting in incorporation of the unnatural amino acid into a polypeptide encoded by the mRNA (Kohrer et al., 2001, Proc. Natl. Acad. Sci., USA, 98:14310; and Kohrer et al., 2004, Nuc. Acid. Res., 32:6200; both of which are incorporated herein by reference). However, as in the case of siRNA delivery, existing methods of delivering tRNAs to cells result in variable levels of delivery, complicating efforts to analyze such proteins and their effects on cells.

Fluorinated lipids may be conjugated to tRNAs, e.g. suppressor tRNAs, to achieve the synthesis of proteins that incorporate an unnatural amino acid with which the tRNA is aminoacylated. The analysis of proteins that incorporate one or more unnatural amino acids has a wide variety of applications. For example, incorporation of amino acids modified with detectable (e.g., fluorescent) moieties can allow the study of protein trafficking, secretion, etc., with minimal disturbance to the native protein structure. Alternatively or additionally, incorporation of reactive moieties (e.g., photoactivatable and/or cross-linkable groups) can be used to identify protein interaction partners and/or to define three-dimensional structural motifs. Incorporation of phosphorylated amino acids such as phosphotyrosine, phosphothreonine, or phosphoserine, or analogs thereof, into proteins can be used to study cell signaling pathways and requirements.

In some embodiments, the functional RNA is a ribozyme. A ribozyme is designed to catalytically cleave target mRNA transcripts may be used to prevent translation of a target mRNA and/or expression of a target (see, e.g. PCT publication WO 90/11364; and Sarver et al., 1990, Science 247:1222; both of which are incorporated herein by reference).

In some embodiments, endogenous target gene expression may be reduced by targeting deoxyribonucleotide sequences complementary to the regulatory region of the target gene (i.e., the target gene's promoter and/or enhancers) to form triple helical structures that prevent transcription of the target gene in target muscle cells in the body (see generally, Helene, 1991, Anticancer Drug Des. 6:569; Helene et al., 1992, Ann, N. Y Acad. Sci. 660:27; and Maher, 1992, Bioassays 14:807; all of which are incorporated herein by reference).

RNAs, including functional classes of RNAs described herein, can be prepared according to any available technique including, but not limited to chemical synthesis, enzymatic synthesis, enzymatic or chemical cleavage of a longer precursor, etc. Methods of synthesizing RNA molecules are known in the art (see, e.g. Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in molecular biology, v. 288 (Clifton, N.J.) Totowa, N.J.: Humana Press, 2005). RNAi agents such as siRNAs are commercially available from a number of different suppliers. Pre-tested siRNAs targeted to a wide variety of different genes are available, e.g., from Ambion (Austin, Tex.), Dharmacon (Lafayette, Colo.), Sigma-Aldrich (St. Louis, Mo.).

Synthetic RNAs such as RNAi agents can include naturally occurring nucleotides, and may include one or more nucleotide analogs or have a structure that otherwise differs from that of a naturally occurring nucleic acid. U.S. Pat. Nos. 6,403,779; 6,399,754; 6,225,460; 6,127,533; 6,031,086; 6,005,087; 5,977,089; and references therein (incorporated herein by reference) disclose a wide variety of specific nucleotide analogs and modifications that may be used in a functional RNA. See Crooke, S. (ed.) Antisense Drug Technology: Principles, Strategies, and Applications (1st ed), Marcel Dekker; ISBN: 0824705661; 1st edition (2001) and references therein. For example, 2′-modifications include halo, alkoxy and allyloxy groups. In some embodiments, the 2′-OH group is replaced by a group selected from H, OR_{y}, R_{y}, halo, SH, SRy, NH₂, NH_{y}, N(R_{y})₂ or CN, wherein R_{y} is C₁-C₆ alkyl, alkenyl or alkynyl and halo is F, Cl, Br or I. Examples of modified linkages include phosphorothioate and 5′-N-phosphoramidite linkages.

Nucleic acids can include nucleotide analogs, modified backbones, or non-naturally occurring internucleoside linkages. Nucleic acids containing one or more of these features can effectively mediate RNAi provided that they have contain a guide strand with a nucleobase sequence that is sufficiently complementary to the target gene. In some cases, RNAi agents containing such modifications display improved properties relative to nucleic acids consisting only of naturally occurring nucleotides. For example, the structure of an siRNA may be stabilized by including nucleotide analogs at the 3′ end of one or both strands order to reduce digestion, e.g. by exonucleases.

Modified nucleic acids need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of an RNAi agent such that the target-specific silencing activity is not substantially affected. The modified region may be at the 5′-end and/or the 3′-end of one or both strands. For example, modified siRNAs in which approximately 1 to approximately 5 residues at the 5′ and/or 3′ end of either of both strands are nucleotide analogs and/or have a backbone modification have been employed. The modification may be a 5′ or 3′ terminal modification. One or both nucleic acid strands of an active RNAi agent may comprise at least 50% unmodified RNA, at least 80% modified RNA, at least 90% unmodified RNA, or 100% unmodified RNA. In certain embodiments, one or more of the nucleic acids in an RNAi agent comprises 100% unmodified RNA within the portion of the guide strand that participates in duplex formation with a target nucleic acid.

RNAi agents may, for example, contain a modification to a sugar, nucleoside, or internucleoside linkage such as those described in U.S. Patent Publications 2003/0175950, 2004/0192626, 2004/0092470, 2005/0020525, and 2005/0032733 (all of which are incorporated herein by reference). Studies describing the effect of a variety of different siRNA modifications have been reviewed (see Manoharan, 2004, Curr. Opin. Chem. Biol., 8:570; incorporated herein by reference). The present invention encompasses the use of an RNAi agent having any one or more of the modification described therein. For example, a number of terminal conjugates, e.g., lipids such as cholesterol, lithocholic acid, aluric acid, or long alkyl branched chains have been reported to improve cellular uptake. Analogs and modifications may be tested using, e.g. using assays such as Western blots, immunofluorescence, or any appropriate assay known in the art, in order to select those that effectively reduce expression of target genes and/or result in improved stability, uptake, etc.

Small Molecules

In some embodiments, a fluorinated lipid is conjugated to a small molecule and/or organic compound. In some embodiments, the small molecule is a small molecule that binds to a target molecule (e.g., a peptide, such as an enzyme) and binds with sufficient affinity such that the fluorinated lipid compound, when associated with the target molecule, delivers the target molecule into a cell. In some embodiments, the small molecule is biotin, and the target molecule is a molecule comprising an avidin or streptavidin peptide. In some embodiments, the small molecule is an inhibitor of a peptide (e.g., an enzyme).

In some embodiments, a fluorinated lipid is conjugated to a small molecule with pharmaceutical activity, e.g., a clinically-used drug. Fluorinated lipids conjugated to pharmaceutically active small molecules may be used in methods of delivering the small molecules to cells (e.g., in vivo, in therapeutic methods). In some embodiments, the drug is an antibiotic, anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, beta-adrenergic blocking agent, diuretic, cardiovascular active agent, vasoactive agent, non-steroidal anti-inflammatory agent, etc.

In some embodiments, anti-cancer agents are selected from approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. Additionally, the present invention also encompasses the use of certain cytotoxic or anticancer agents currently in clinical trials and which may ultimately be approved by the FDA (including, but not limited to, epothilones and analogues thereof and geldanamycins and analogues thereof). For a more comprehensive discussion of updated cancer therapies see, www.nci.nih.gov, a list of the FDA approved oncology drugs at www.fda.gov/cder/cancer/druglistframe.htm, and The Merck Manual, Seventeenth Ed. 1999, the entire contents of which are hereby incorporated by reference.

In some embodiments, the agent is other than methotrexate.

In some embodiments, the agent for delivery is a mixture of pharmaceutically active agents.

Peptides

In some embodiments, a fluorinated lipid is conjugated to a peptide. In certain embodiments, peptides range from about 5 to about 40, about 10 to about 35, about 15 to about 30, or about 20 to about 25 amino acids in size. In certain embodiments, a peptide as at least 40 amino acids (e.g., 45, 50, 60, 70, 80, 90, 100, 150, 200, 250, or more amino acids). Peptides from panels of peptides comprising random sequences and/or sequences which have been varied consistently to provide a maximally diverse panel of peptides may be used.

The terms “polypeptide” and “peptide” are used interchangeably herein. Peptides may contain L-amino acids, D-amino acids, or both and may contain any of a variety of amino acid modifications or analogs known in the art. Useful modifications include, e.g., terminal acetylation, amidation, etc.

In some embodiments, a fluorinated lipid is conjugated to an antibody. Such conjugation may be covalent or non-covalent. In some embodiments, antibodies may include, but are not limited to, polyclonal, monoclonal, chimeric (i.e. “humanized”), single chain (recombinant) antibodies. In some embodiments, antibodies may have reduced effector functions and/or bispecific molecules. In some embodiments, antibodies may include Fab fragments and/or fragments produced by a Fab expression library. In some embodiments, the antibody is a therapeutic antibody.

Carbohydrates

In some embodiments, an agent for delivery to a cell is a carbohydrate (e.g., a natural or synthetic carbohydrate). The carbohydrate may also be a derivatized natural carbohydrate. In certain embodiments, the carbohydrate may be a simple or complex sugar. In certain embodiments, the carbohydrate is a monosaccharide, including but not limited to glucose, fructose, galactose, and ribose. In certain embodiments, the carbohydrate is a disaccharide, including but not limited to lactose, sucrose, maltose, trehalose, and cellobiose. In certain embodiments, the carbohydrate is a polysaccharide, including but not limited to cellulose, microcrystalline cellulose, hydroxypropyl methylcellulose (HPMC), methylcellulose (MC), dextrose, dextran, glycogen, xanthan gum, gellan gum, starch, and pullulan. In certain embodiments, the carbohydrate is a sugar alcohol, including but not limited to mannitol, sorbitol, xylitol, erythritol, maltitol, and lactitol.

### Cells

The fluorinated lipid compositions and methods described herein can be used to deliver agents to any eukaryotic cell of interest. In certain embodiments, a cell is a mammalian cell. Cells may be of human or non-human origin. For example, they may be of mouse, rat, or non-human primate origin. A cell can be of any cell type. Exemplary cell types include, but are not limited to, endothelial cells, epithelial cells, neurons, hepatocytes, myocytes, chondrocytes, osteoblasts, osteoclasts, lymphocytes, macrophages, neutrophils, fibroblasts, keratinocytes, etc. Cells can be primary cells, immortalized cells, transformed cells, terminally differentiated cells, stem cells (e.g. adult or embryonic stem cells, hematopoietic stem cells), somatic cells, germ cells, etc. Cells can be wild type or mutant cells, e.g., they may have a mutation in one or more genes. Cells may be quiescent or actively proliferating. Cells may be in any stage of the cell cycle. In some embodiments, cells may in the context of a tissue. In some embodiments, cells may be in the context of an organism.

Cells can be normal cells or diseased cells. In certain embodiments, cells are cancer cells, e.g. they originate from a tumor or have been transformed in cell culture (e.g. by transfection with an oncogene). In certain embodiments, cells are infected with a virus or other infectious agent. A virus may be, e.g. a DNA virus, RNA virus, retrovirus, etc. For example, cells can be infected with a human pathogen such as a hepatitis virus, a respiratory virus, human immunodeficiency virus, etc.

Cells may have been experimentally manipulated to overexpress one or more genes of interest.

Cells can be cells of a cell line. Exemplary cell lines include HeLa, CHO, COS, BHK, NIH-3T3, HUVEC, etc. For an extensive list of mammalian cell lines, those of ordinary skill in the art may refer to the American Type Culture Collection catalog (ATCC®, Manassas, Va.).

Cells can be sorted based on the presence of a characteristic. In some embodiments, cells are exposed to a fluorinated lipid compound described herein, and sorted based on the presence of a characteristic that correlates with internalization of the fluorinated lipid compound in the cell. Methods for analyzing and separating cells are described, e.g., in PCT Publication WO 07/67733 (incorporated herein by reference).

### Pharmaceutical Compositions

Fluorinated lipid compositions comprising a linker and an agent for delivery can be provided as pharmaceutical compositions. In some embodiments, the present invention provides pharmaceutical compositions comprising fluorinated lipid compounds as described herein and one or more pharmaceutically acceptable excipients. Such pharmaceutical compositions may optionally comprise one or more additional therapeutically-active substances. In accordance with some embodiments, a method of administering pharmaceutical compositions comprising a fluorinated lipid compound to a subject in need thereof is provided. In some embodiments, compositions are administered to humans. For the purposes of the present disclosure, the phrase “active ingredient” generally refers to fluorinated lipid compound as described herein.

Pharmaceutical compositions provided herein include pharmaceutical compositions which are suitable for ethical administration to humans, as well as compositions suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100% (w/w) active ingredient.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum™. [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween™20], polyoxyethylene sorbitan [Tween™60], polyoxyethylene sorbitan monooleate [Tween™80], sorbitan monopalmitate [Span™40], sorbitan monostearate [Span™60], sorbitan tristearate [Span™65], glyceryl monooleate, sorbitan monooleate [Span™80]), polyoxyethylene esters (e.g. polyoxyethylene monostearate [Myrj™45], polyoxyethylene hydrogenated castor oil, polyethoxylated castor oil, polyoxymethylene stearate, and Solutol™), sucrose fatty acid esters, polyethylene glycol fatty acid esters (e.g. Cremophor™), polyoxyethylene ethers, (e.g. polyoxyethylene lauryl ether [Brij™30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic™F 68, Poloxamer™188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum™), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus™, Phenonip™, methylparaben, Germall™115, Germaben™II, Neolone™, Kathon™, and/or Euxyl™.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such an Cremopho™, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants and/or patches. Generally, the active ingredient is admixed under sterile conditions with a pharmaceutically acceptable excipient and/or any needed preservatives and/or buffers as may be required. Additionally, the present invention contemplates the use of transdermal patches, which often have the added advantage of providing controlled delivery of a compound to the body. Such dosage forms may be prepared, for example, by dissolving and/or dispensing the compound in the proper medium. Alternatively or additionally, the rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and the active ingredient may constitute 0.1% to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide the active ingredient in the form of droplets of a solution and/or suspension. Such formulations may be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 nm to about 200 nm.

The formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of the active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 nm to about 200 nm, and may further comprise one or more of the additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this invention.

General considerations in the formulation and/or manufacture of pharmaceutical agents may be found, for example, in Remington: The Science and Practice of Pharmacy 21st ed., Lippincott Williams & Wilkins, 2005.

### Administration to a Subject

Fluorinated lipid compounds, according to a method of the present invention, may be administered to a subject using any amount and any route of administration effective for treating a disease, disorder, and/or condition. The exact amount required will vary from subject to subject, depending on the species, age, and general condition of the subject, the severity of the infection, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Pharmaceutical compositions may be administered to animals, such as mammals (e.g., humans, domesticated animals, cats, dogs, mice, rats, etc.). In some embodiments, pharmaceutical compositions are administered to humans. The pharmaceutical compositions in accordance with the present invention may be administered by any route. In some embodiments, pharmaceutical compositions of the present invention are administered by a variety of routes, including oral, intravenous, intramuscular, intra-arterial, intramedullary, intrathecal, subcutaneous, intraventricular, transdermal, interdermal, rectal, intravaginal, intraperitoneal, topical (e.g. by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter. In some embodiments, pharmaceutical compositions are administered by systemic intravenous injection, regional administration via blood and/or lymph supply, and/or direct administration to an affected site (e.g. a therapeutic implant, such as a hydrogel). In specific embodiments, thermally-responsive conjugates in accordance with the present invention and/or pharmaceutical compositions thereof may be administered intravenously. In specific embodiments, fluorinated phospholipid compounds in accordance with the present invention and/or pharmaceutical compositions thereof may be administered intraperitoneally. In specific embodiments, fluorinated phospholipid compounds in accordance with the present invention and/or pharmaceutical compositions thereof may be administered intrathecally. In specific embodiments, fluorinated phospholipid compounds in accordance with the present invention and/or pharmaceutical compositions thereof may be administered intratumorally. In specific embodiments, fluorinated phospholipid compounds in accordance with the present invention and/or pharmaceutical compositions thereof may be administered intramuscularly. In specific embodiments, fluorinated phospholipid compounds in accordance with the present invention and/or pharmaceutical compositions thereof may be administered via vitreal administration. In specific embodiments, fluorinated phospholipid compounds in accordance with the present invention and/or pharmaceutical compositions thereof may be administered via a portal vein catheter. In specific embodiments, fluorinated phospholipid compounds in accordance with the present invention and/or pharmaceutical compositions thereof may be immobilized into a hydrogel for controlled long-term release of fluorinated phospholipid compounds. However, the invention encompasses the delivery of fluorinated phospholipid compounds and/or pharmaceutical compositions thereof by any appropriate route taking into consideration likely advances in the sciences of drug delivery.

In general the most appropriate route of administration will depend upon a variety of factors including the nature of the agent (e.g., its stability in the environment of the gastrointestinal tract), the condition of the patient (e.g. whether the patient is able to tolerate oral administration), etc. The invention encompasses the delivery of the pharmaceutical compositions by any appropriate route taking into consideration likely advances in the sciences of drug delivery.

In certain embodiments, compositions in accordance with the invention may be administered parenterally at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

Pharmaceutical compositions in accordance with the present invention may be administered either alone or in combination with one or more other therapeutic agents. By “in combination with,” it is not intended to imply that the agents must be administered at the same time and/or formulated for delivery together, although these methods of delivery are within the scope of the invention. Compositions can be administered concurrently with, prior to, or subsequent to, one or more other desired therapeutics or medical procedures. In general, each agent will be administered at a dose and/or on a time schedule determined for that agent. In some embodiments the invention encompasses the delivery of pharmaceutical compositions in combination with agents that may improve their bioavailability, reduce and/or modify their metabolism, inhibit their excretion, and/or modify their distribution within the body.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer in accordance with the invention may be administered concurrently with another anticancer agent), or they may achieve different effects (e.g. control of any adverse effects).

Fluorinated lipid compounds and/or pharmaceutical compositions in accordance with the present invention may be administered alone and/or in combination with other fluorinated lipid compounds and/or agents for treatment of a disease, disorder, or condition. In will further be appreciated that therapeutically active agents utilized in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually.

### Applications

Methods in accordance with the invention may be used to deliver agents to cells (e.g., cells within specific tissues). In some embodiments, a fluorinated lipid compound is used to deliver a nucleic acid, peptide, or small molecule (e.g., for a diagnostic or therapeutic use).

In certain embodiments, a fluorinated lipid compound is formulated with one or more agents that mediate controlled release of the compound.

The invention encompasses in vivo applications of the compositions and methods described herein. In certain embodiments, a composition comprising a fluorinated lipid compound, e.g., a fluorinated lipid conjugated to a linker and a nucleic acid is administered to a subject.

In some embodiments, following administration to a subject, the fluorinated lipid compound is detected, thereby providing an indication of the distribution and/or uptake of the compound by various cells, tissues, organs, etc., and optionally providing an indication of the activity of the agent in such cells, tissues, organs, etc. In some embodiments, the compound is conjugated to a detectable agent. Detection can take place at any suitable time following administration. In some embodiments, a tissue sample (e.g., a tissue section) is obtained from the subject and examined microscopically. Alternately, individual cells can be isolated from the subject and examined, sorted, or further processed. In vivo imaging techniques such as fluorescence imaging can be employed to detect fluorinated lipid compounds in a living subject (Gao et al., 2004, Nat. Biotechnol., 22:969; incorporated herein by reference). Conventional immunostaining or other techniques can be employed, e.g. to detect an agent in vivo, or to evaluate its efficacy.

### Kits

The invention provides a variety of kits for conveniently and/or effectively making or carrying out methods of the present invention. Inventive kits typically a fluorinated lipid compound including a linker and an agent for delivery to a cell. In some embodiments, a kit includes more than one type of fluorinated lipid compound. Typically kits will include sufficient amounts of a compound to allow a user to use the compound multiple times (e.g., for multiple nucleic acid transfections, or for multiple treatments of a subject(s) and/or to perform multiple experiments. In some embodiments, kits are supplied with fluorinated lipid compounds that include one or more agents for delivery to a cell, wherein the agents have been specified by the purchaser.

In some embodiments, a kit includes a fluorinated lipid linked to a linker, wherein the linker has a reactive moiety (e.g., a thiol-reactive moiety). The kit can further include a reagent for modifying an agent so as to be reactive with the linker. In some embodiments, the kit comprises a compound of formula:

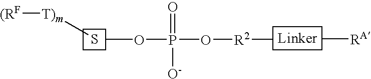

wherein:

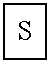

is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{A′} is hydrogen or an optionally substituted thiol-reactive, amine-reactive, or hydroxyl-reactive moiety;
each occurrence of n is an integer from 0 to 30, inclusive, wherein at least one occurrence of n is non-zero; and
m is an integer from 1 to 2, inclusive, wherein m is 1 when

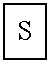

is a covalent bond.

In certain embodiments, the compound is covalently linked to a therapeutic agent as described herein.

In some embodiments, the kit comprises a compound of formula:

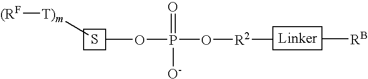

wherein:

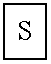

is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{B} is an optionally substituted moiety capable of forming a non-covalent interaction with a therapeutic agent;
each occurrence of n is an integer from 0 to 30, inclusive, wherein at least one occurrence of n is non-zero; and
m is an integer from 1 to 2, inclusive, wherein m is 1 when

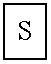

is a covalent bond.

In certain embodiments, the phospholipid is non-covalently linked to a therapeutic agent as described herein.

Kits may include additional components or reagents. For example, kits may comprise one or more control compounds, e.g., positive control (fluorinated lipid compounds known to deliver a particular agent) and negative control (fluorinated lipid compounds known not to deliver a particular agent). Other components of inventive kits may include cells, cell culture media, tissue, and/or tissue culture media.

Kits may include instructions for use. For example, instructions may inform the user of the proper procedure by which to prepare a pharmaceutical composition comprising fluorinated lipid compounds and/or the proper procedure for administering the pharmaceutical composition to a subject.

Kits can include one or more vessels or containers so that certain of the individual components or reagents may be separately housed. Kits can include a means for enclosing the individual containers in relatively close confinement for commercial sale, e.g., a plastic box, in which instructions, packaging materials such as styrofoam, etc., may be enclosed.

In some embodiments, inventive kits include one or more fluorinated lipid compounds including at least one fluorinated phospholipid, a linker, and an agent for delivery to a cell. In some embodiments, such a kit is used for delivering an agent (e.g., a nucleic acid) to a cell in vitro. In some embodiments, a kit is used in the treatment, diagnosis, and/or prophylaxis of a subject suffering from and/or susceptible to a disease, condition, and/or disorder. In some embodiments, a kit includes a syringe, needle, applicator, etc. for administration to a subject; and instructions for use.

### Exemplification

### General

Wide-field Microscopy: All images were taken on a Nikon IX70-based DeltaVision RT restoration microscope equipped with an Olympus 60× N.A. 1.40 Plan-Apochromat oil immersion lens, a 3D-motorized stage, appropriate filter sets (Chroma), photometrics CoolSNAP HQ CCD-camera. Optical sections were acquired every 0.4 mm for the complete 3D cellular volume. All of the images were taken within the linear range below the pixel saturation value of the camera. Images of 3D data sets were corrected for any fluctuation in mercury lamp and restored by using an iterative constrained deconvolution algorithm based on empirically measured point-spread function by using the built-in softWORX image processing package (Applied Precision Inc.).

### Example 1

### Fluorinated Lipids Permit Facile Passage of Macromolecules into Living Cells

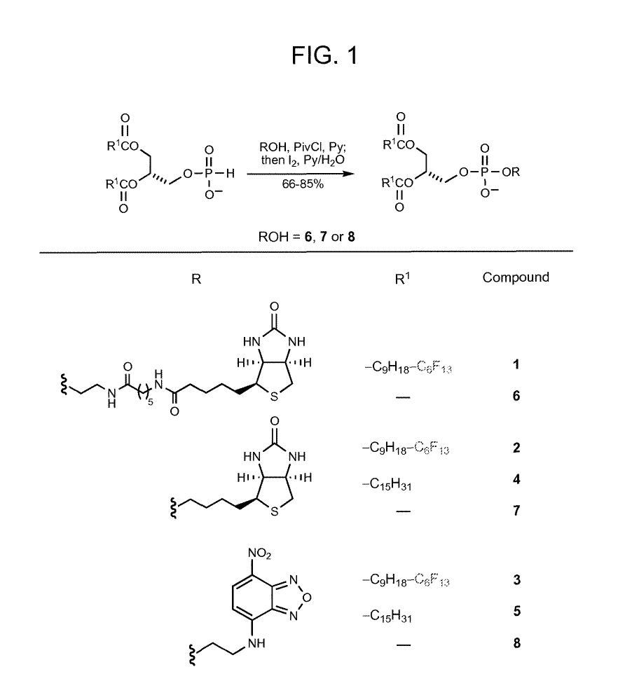

In order to investigate the ability of fluorinated lipids to act as macromolecule transport agents, compounds 1-5 shown in FIG. 1 were designed and synthesized (Scheme 1). Agents 1, 3 and 5 are derivatives of 1,2-dipalmitoyl-sn-glycero-3-phosphoethanolamine (DPPE) while 2 and 4 are related phosphodiesters. Preparation of conjugates from the respective H-phosphonates followed procedures described in Liu et al., J. Am. Chem. Soc. 2006, 128, 3638. These molecules contain either all hydrocarbon (4 and 5) or partially fluorinated lipid chains (1-3) and the head groups are adorned with different linkers attached to biotin (1, 2 and 4) or to the fluorophore 7-nitrobenz-2-oxa-1,3-diazole (NSD, 3 and 5). The agents were deemed non-toxic to cells in culture as incubation of HeLa cells with 2-5 did not show any difference in counts after five days when compared to controls. In addition, the complex of 2 and 4 with avidin conjugated to fluorescein isothiocyanate (AF, Kₐ to biotin ˜10¹⁵ M⁻¹) also had little effect on the growth profiles (data not shown).

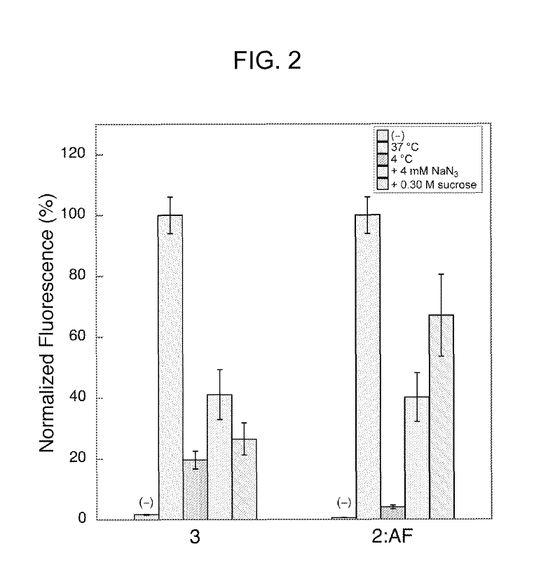

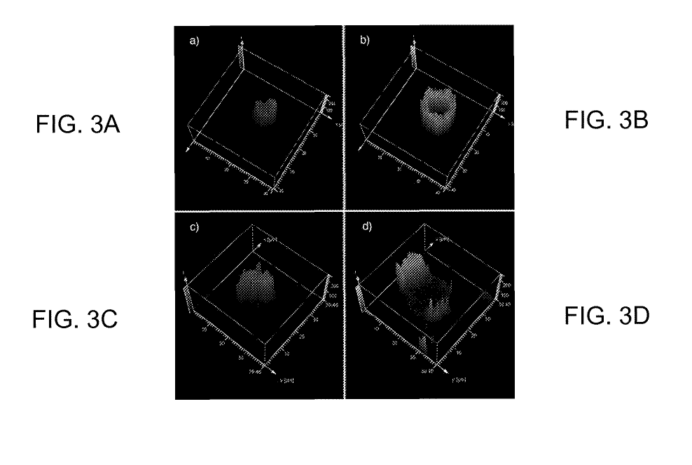

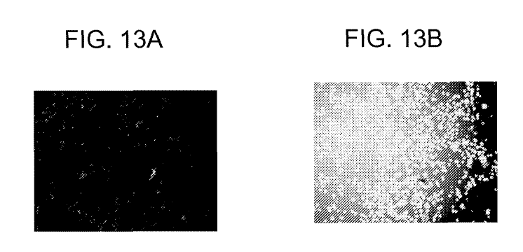

Incubation of HeLa cells with 3 at 37° C. resulted in intensely fluorescent cells as evinced by counting on a fluorescence plate reader (FIG. 2). The increase in fluorescence was concentration dependent. Further inspection by microscopy revealed that the emanating fluorescence was distributed both on the cellular surface and in the interior. Similar experiments with 8 did not result in any cellular fluorescence (See FIGS. 13A and 13B, which show cells incubated with 8 (FIG. 13A) and 3 (FIG. 13B). Jurkat and HL60 cells when treated similarly with 3 exhibited comparable levels of fluorescence suggesting that the process is general across many cell lines. Localization of 3 was further probed in HeLA cells using confocal fluorescence microscopy (FIGS. 3a and 3b) and wide-field microscopy followed by deconvolution algorithms to reconstruct the 3D distribution of fluorescent molecules in the sample.

Internalization of lipids was energy dependent suggesting that it is facilitated by endocytosis (Boonyarattanakalin et al., J. Am. Chem. Soc. 2004, 126, 16379). When incubations of HeLa cells with 3 or the complex 2:AF were carried out at 4° C., fluorescence from the cells decreased dramatically. Indeed, only 20 and 4% of fluorescence was detectable when compared to the experiment at 37° C. for 3 and the 2:AF complex respectively (FIG. 2). Furthermore, addition of sodium azide, an ATP depleting poison and a known inhibitor of endocytosis, also resulted in a ˜2.5-fold decrease in fluorescence. These experiments implicated endocytosis as the primary mediator of transport of the exogenous materials (Schmid and Carter, J. Cell Biol. 1990, 111, 2307). It was further investigated whether endocytosis was orchestrated via a particular pathway. Participation of clathrin coated pits is frequently invoked in endocytic events and it can be disrupted by incubation under hypertonic conditions (300-450 mM sucrose)(Heuser and Anderson, J. Cell Biol. 1989, 108, 389). Indeed, when HeLa cells were treated with 3 or the 2:AF complex under such conditions, the fluorescence from the cells was diminished by 74 and 33% respectively. These results were further corroborated with fluorescence microscopy of the resultant cells (see below).

The ability of the transport agents to deliver macromolecules across membranes was investigated by incubation of Hela cells with preformed complexes 1:AF, 2:AF or 4:AF. The cells were pelleted by centrifugation twice and washed with PBS, re-suspended and then examined by microscopy or fluorescence counting. All agents were effective at ferrying AF into the cell. In contrast, neither AF nor the AF:biotin complex were by themselves able to traverse the membrane resulting in cells that were minimally fluorescent. The binding affinity of lipid-biotin conjugates to AF is several orders of magnitude less tight than biotin (Hussey and Peterson, J. Am. Chem. Soc. 2002, 124, 6265; Powers et al., Biotechnol. Frog. 1992, 8, 436). Indeed, when free biotin (≧5 eq, 1 h) was allowed to equilibrate with 2:AF prior to incubation with HeLa cells, the cells only exhibited background fluorescence.

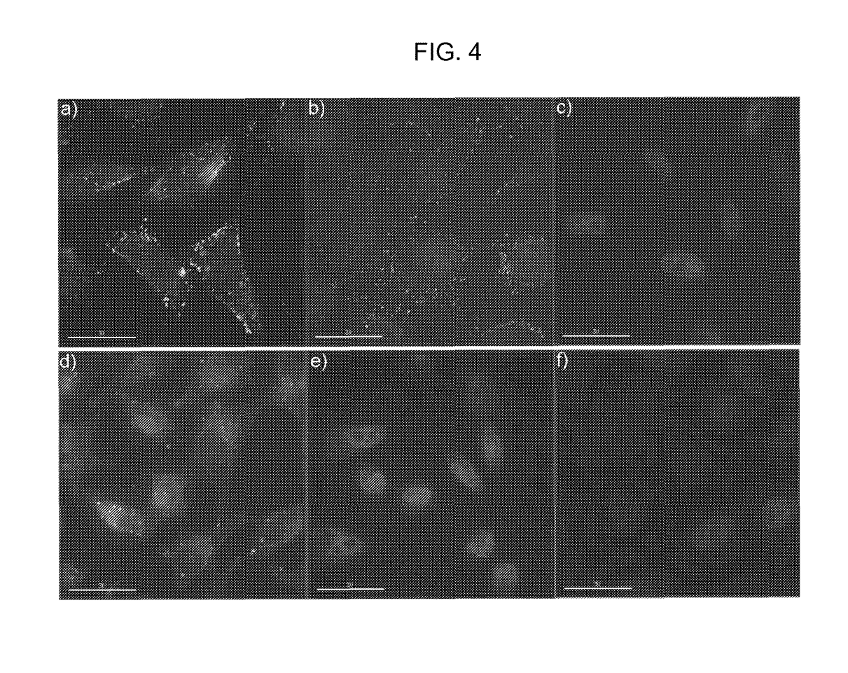

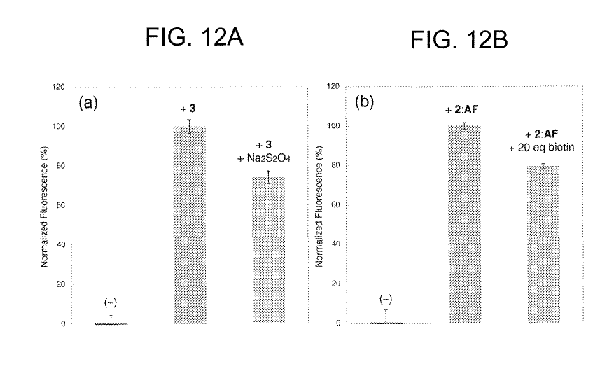

In order to assess what fraction of 3 or the complex 2:AF resides on the outer leaflet of the plasma membrane, cells were subjected to reduction by dithionite (in the case of 3; see FIG. 12a) or to exchange with free biotin (in the case of the 2:AF complex; see FIG. 12b). Upon reduction with 5 mM dithionite, a reagent known to extinguish NBD fluorescence, 26% of the fluorescence from the cells was lost. Biotin exchange for 1 h resulted in a similar (21%) loss of fluorescence in the case of the 2:AF treated cells. Assuming NBD fluorescence is not affected by the environment, upon treatment with a 100 μM solution, 10¹⁰ molecules were internalized and 2×10⁹ molecules were present on the surface of each cell and available for reduction. The localization of synthetic constructs and macromolecular cargo inside cells was investigated using microscopy done in the presence of a nucleus specific dye, 4′,6-diamidino-2-phenylindole (DAPI). As the overlay images show in FIG. 4, light emitted by 3 and AF originates from the cytoplasmic region and the agents are excluded entirely from the nucleus.

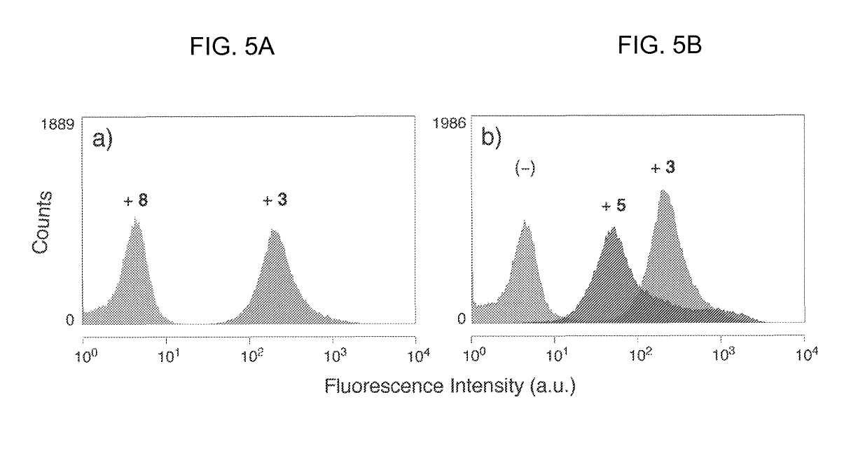

In general, the fluorinated lipid constructs 2 and 3 were more efficient in transport and uptake as compared to their hydrocarbon counterparts 4 and 5. As judged from fluorescence counting, this difference was 2.6 fold. This difference in efficiency of uptake was also confirmed using flow cytometry (FIGS. 5a and 5b). While the hydrocarbon lipids conferred a 13-fold increase in mean fluorescence intensity of cells over background, the cells treated with the fluorinated congeners were intensely fluorescent with a 63-fold increase in mean fluorescence (a difference of 4.8-fold). These results demonstrate the superior ability of the fluorinated lipids to trigger and act as participants in the endocytic events. In summary, we have discovered a class of new macromolecular transport agents capable of entering living cells through endocytosis. They are furthermore able to facilitate the entry of noncovalently bound proteins. The delivery agents were non-toxic and were distributed both on the surface and in the cytoplasm of cells but not in the nucleus. Fluorinated lipids were found to be superior in facilitating transport in this manner. A large number of molecules can be displayed on the cell surface using this methodology (˜10⁹). This study paves the way for clustered display of ligands on cell surfaces and intracellular delivery of macromolecules for imaging and therapeutic applications. Studies along these lines are in progress in our laboratories.

General Procedure for Incubation of Cells with Compounds: Cells were cultured in DMEM/RPMI medium supplemented with 10% fetal bovine serum and 1% penicillin/streptomycin. Cells were suspended in phosphate buffered saline (PBS), incubated with an ethanolic solution of lipid conjugates for 1-2 h at 4 or 37° C. to give a final concentration in each well of 0.01-100 μM in a total volume of 100 μL (10% EtOH). After the incubation period, cells were pelleted by centrifugation and washed (2×) and resuspended in 100 μL PBS and analyzed.

ATP Depletion: Cells were pretreated with 4 mM NaN₃/0.8 mM NaF in 100 μL PBS for 30 m at 37° C. The cells were then washed with PBS (2×100 μL) and incubated with 100 μM ethanolic solutions of lipids for 2 h (total volume 100 μL, 10% EtOH). Analysis was carried out as described previously.

Incubation under Hypertonic Conditions: Cells were pretreated with 0.3 M sucrose for 30 min at 37° C. in PBS, and were then incubated with lipid solutions. Analysis was carried out as described previously.

Toxicity Experiment: HeLa cells (˜1.0×10⁶ cells/mL) were incubated with 100 μM solutions of 2-5, 2:AF or 5:AF for 5 d. These experiments were carried out in a 6-well plate and viable cells were counted every 24 h.

Fluorescence Assay: After the incubation period, HeLa cells in PBS were analyzed using an Infinite™ 200 series microtiterplate reader (Tecan Systems, Inc.). All experiments were carried out in 96 well plates with six replicates. The plates were pretreated with 1% BSA for 12 h at 4° C. to minimize non-specific binding.

Fluorescence Microscopy: HeLa cells were imaged using a modified Olympus IX81 Motorized Inverted Microscope (Optical Analysis Co., Nashua, N.H.). Images were acquired using a CCD camera (Orca-ER, Hamamatsu, Japan) with λ_{exc}=494 nm and λ_{exc}=530 nm with a 1 ms exposure (Filter set 31003, Chroma Technology Corp, Rockingham, Vt., USA).

Flow cytometry: Treated HeLa cells (˜3×10⁴) were suspended in PBS and analyzed using flow cytometry on a MoFlo™ (Dako A/S, Glostrup, Denmark) instrument.

Confocal Fluorescence Microscopy: Imaging was performed on a Zeiss LSM 510 META Laser Scanning Microscope using an Ar_{458 nm} excitation laser.

### Example 2

### Synthesis of Fluorinated Lipids

General Procedures. Flash column chromatography was performed on Kieselgel 60 silica gel (230-240 mesh, EM Science) using standard litertaure procedures. Analytical thin layer chromatography was performed using E. Merck silica gel Kieselgel 60 F₂₅₄ (0.25 mm) plates. Compounds were visualized by UV light, exposure to iodine vapour or by staining with a ninhydrin solution followed by heating. Reagents and solvents were of reagent grade or better and were obtained from Aldrich Chemical Co., Fluka Chemie AG, Fluorochem USA, Lancaster Synthesis or Novabiochem Corp. Deuterated solvents were obtained from Cambridge Isotope Laboratories.

Nuclear magnetic resonance spectra were recorded on a Bruker AM-300 or a Bruker DPX-300 instrument in standard deuterated solvents. ¹⁹F NMR spectra were measured using CFCl₃ (δ=0) for organic solvents and CF₃CO₂H (δ=−76.50) for D₂O as the internal standards. Electrospray mass spectra (ESI-MS) were recorded using a ThermoQuest LCQ Deca.

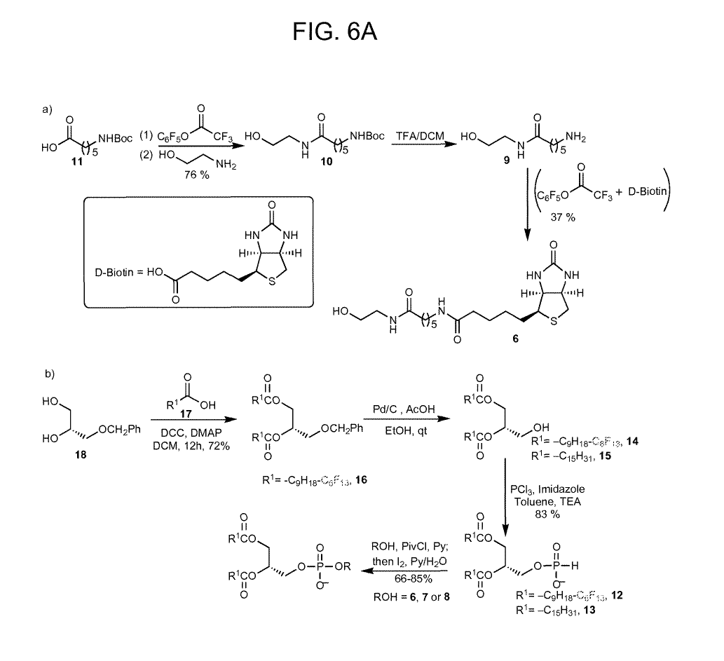

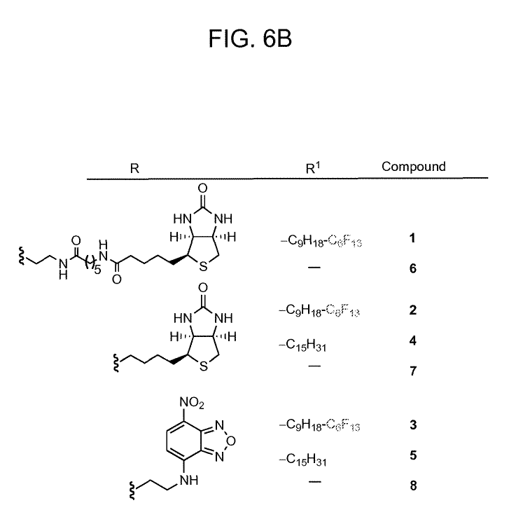

Compound numbers in this section refer to compounds shown in FIGS. 6A and 6B.

Compound 16. A solution of DMAP (118 mg, 0.97 mmol), 3-O-benzyl-sn-glycerol (18) (0.50 g, 2.74 mmol), and 17 (Yoder et al., J. Am. Chem. Soc. 2007, 129, 9037-9043) (5.38 g, 10.9 mmol) in anhydrous CH₂Cl₂ (18 mL) at 0° C. was added dropwise over 15 min to dicyclohexylcarbodiimide (DCC) (2.26 g, 10.9 mmol) in 13 mL of anhydrous CH₂Cl₂. The reaction was stirred for 1 h at 0° C., and then for 16 h at rt. Volatiles were removed under reduced pressure and the product (16) was purified by flash chromatography using 20% EtOAc/hexane yielding 2.60 g (72%). ¹H NMR (CDCl₃, 300 MHz) δ 7.32 (m, 5H), 5.25 (m, 1H), 4.54 (dd, J=12.0 Hz, J=3.9 Hz, 2H), 4.35 (dd, J=8.1 Hz, J=3.9 Hz, 1H), 4.18 (dd, J=6.3 Hz, J=5.4 Hz, 1H), 3.59 (d, J=5.4 Hz, 2H), 2.29 (m, 4H), 2.01 (m, 4H), 1.60 (m, 8H), 1.29 (m, 20H); ¹³C NMR (CDCl₃, 75.5 MHz) δ 173.8, 173.5, 138.1, 128.8, 128.2, 128.0, 73.7, 70.4, 68.7, 63.1, 34.6 (d, J=16.5 Hz), 32.9, 31.2 (t, J=22.3 Hz), 29.6, 29.5, 29.4, 25.3, 25.2, 23.8, 20.5; ¹⁹F NMR (CDCl₃, 282.6 MHz) δ −81.3 (m, 6F), −114.9 (m, 4F), −122.4 (m, 4F), −123.4 (m, 4F), −124.1 (m, 4F), −126.6 (m, 4F); ESI-MS (spray voltage: 4.5 kV, capillary temp: 250° C., capillary voltage: 40 V, tube lens offset: 250 V) calcd. for [C₄₂H₄₈F₂₆O₅.H]⁺ m/z 1126.8, found: m/z 1126.6.

Compound 14. Compound 16 (1.00 g, 0.89 mmol) was dissolved in a mixture of absolute EtOH (15 mL) and glacial AcOH (1.2 mL) containing 10% Pd/C (45 mg) and the mixture was stirred under atmosphere of H₂ at rt for 4 h. The reaction mixture was filtered through celite and the filtrate evaporated under reduced pressure at 25° C. The product was further purified by flash chromatography with 1:5 EtOAc/hexane mixture to give 14 in quantitative yield (0.92 g) as a colorless liquid. In order to prevent 2,3-acyl migration, 14 was stored at −20° C. (Liu et al., J. Am. Chem. Soc. 2006, 128, 3638-3648). ¹H NMR (CDCl₃, 300 MHz) δ 5.08 (m, 1H), 4.32 (dd, J=11.7 Hz, J=4.5 Hz, 1H), 4.23 (dd, J=12.0 Hz, J=5.7 Hz, 1H), 3.75 (d, J=7.8 Hz, 2H), 2.34 (m, 4H), 2.10 (m, 4H), 1.56 (m, 8H), 1.30 (m, 20H); ¹³C NMR (CDCl₃, 75.5 MHz) δ 174.2 (C═O), 173.8 (C═O), 72.5, 62.4, 62.0, 35.3, 34.6 (d, J=14.1 Hz), 31.2 (t, J=22.1 Hz), 29.6, 29.5, 29.4, 25.3, 25.2, 21.0, 20.5; ¹⁹F NMR (CDCl₃, 282.6 MHz) δ −81.3 (m, 6F), −114.9 (m, 4F), −122.4 (m, 4F), −123.4 (m, 4F), −124.1 (m, 4F), −126.6 (m, 4F); ESI-MS (spray voltage: 4.5 kV, capillary temp: 250° C., capillary voltage: 40 V, tube lens offset: 250 V) calcd. for [C₃₅H₄₂F₂₆O₅.Na]⁺ m/z 1059.6, found m/z 1059.3.

General procedure for two-step, one pot amide bond formation. R—CO₂H (D-biotin or 11, 0.97 mmol) was dissolved in 10 mL of anhydrous DMF and Et₃N (405 μL, 2.91 mmol) was added to it. The reaction flask was cooled in an ice bath and 2,3,4,5,6-pentafluorophenyl trifluoroacetate (PFP-TFA) was added (201 μL, 1.17 mmol) dropwise resulting in a purple solution. The reaction mixture was then stirred for an additional 3 h at rt and TLC analysis (5% MeOH in CHCl₃) showed the disappearance of R—CO₂H. Ethanolamine or 9 (0.97 mmol) in 2 mL of DMF was then added to the flask containing PFP-activated acid. The purple solution turned yellow. The reaction mixture was stirred overnight at rt under argon. After removal of solvents under reduced pressure, the resulting residue was subjected to flash column chromatography in 9:1 CHCl₃/MeOH to give a yellowish gummy solid (10: 76% or 6: 37%).

Boc deprotection. To a solution of 10 (0.9 g, 3.3 mmol) in CH₂Cl₂ (16 mL) was added TFA (16 mL). The reaction was stirred at room temperature for 1 h and concentrated in vacuo to give 9 in quantitative yield.

Compound 10. ¹H NMR (CD₃OD, 300 MHz) δ 6.50 (s, 1H), 4.90 (s, 1H), 3.67 (t, J=5.0 Hz, 2H), 3.37 (dd, d, J=10.2 Hz, J=5.4 Hz, 2H), 3.05 (dd, J=13.2 Hz, J=6.6 Hz, 2H), 2.19 (t, J=7.5 Hz, 2H), 1.62 (m, 2H), 1.48-1.34 (m, 13H); ¹³C NMR (CD₃OD, 75.5 MHz) δ 174.7 (C═O), 162.5 (C═O), 79.7, 62.4, 61.9, 60.8, 53.8, 40.7, 36.7, 30.1, 28.8, 26.6; ESI-MS (spray voltage: 4.5 kV, capillary temp: 250° C., capillary voltage: 40 V, tube lens offset: 250 V) calcd. for [C₁₃H₂₆N₂O₄.Na]⁺ m/z 297.3, found m/z 297.4.

Compound 9. ESI-MS calcd. for [C₈H₁₈N₂O₂.H]⁺ m/z 175.2, found m/z 175.1 (Liu et al., J. Am. Chem. Soc. 2006, 128, 3638-3648).

Compound 6. ¹H NMR (CD₃OD, 300 MHz) δ 4.51 (m, 1H), 4.35 (m, 1H), 3.61 (t, J=5.7 Hz, 2H), 3.34 (m, 3H), 3.22 (m, 4H), 2.96 (dd, J=12.8 Hz, J=5.0 Hz, 1H), 2.74 (d, J=12.9 Hz, 1H), 2.23 (m, 4H), 1.70-1.40 (m, 13H); ¹³C NMR (CD₃OD, 75.5 MHz) δ 175.4 (C═O), 174.9 (C═O), 165.1 (C═O), 62.4, 60.6 (2C), 56.1, 41.9, 40.1, 39.2, 35.8, 29.1, 28.8, 28.5, 26.5, 25.9, 25.6; ESI-MS (spray voltage: 4.5 kV, capillary temp: 250° C., capillary voltage: 40 V, tube lens offset: 250 V) calcd. for [C₁₉H₃₂N₄O₄S.H]⁺ m/z 401.5, found m/z 401.4.

Compound 12. Imidazole (1.52 g, 22.3 mmol) was co-evaporated with toluene (10 mL) and dried in vacuo for 1 h. The mixture was dissolved in toluene (18 mL) and cooled to 0° C. PCl₃ (0.43 mL, 4.91 mmol) in toluene (5 mL) and Et₃N (1.95 mL, 14.0 mmol) were added successively, and the reaction mixture stirred for an additional 30 min at 0° C. The reaction mixture was then cooled to −10° C. and a solution of 14 (1.00 g, 0.96 mmol) in toluene (14 mL) and CH₂Cl₂ (4 mL) was added dropwise over a period of 1 h. The resulting mixture was allowed to stir for an additional 1 h at −10° C. and quenched by the addition of water/pyridine (1:4, 30 mL). The organic layer was separated and the aqueous layer was extracted with CHCl₃. The combined organic layers were washed with triethylammonium bicarbonate buffer (TEAB, pH 8.5), dried over MgSO₄, concentrated and purified affording the H-phosphonate 12 as a white gummy solid (0.95 g, 83%) R_{f} 0.36 (20% MeOH in CH₂Cl₂); ¹H NMR (CDCl₃, 300 MHz) δ 5.16 (m, 1H), 4.28 (m, 1H), 4.07 (m, 1H), 3.89 (m, 2H), 3.08 (dd, J=14.7 Hz, J=7.5 Hz, NEt₃H⁺), 2.25 (m, 4H), 1.99 (m, 4H), 1.56 (m, 8H), 1.39 (t, J=7.2 Hz, NEt₃H⁺), 1.27 (m, 20H); ¹³C NMR (CDCl₃, 75.5 MHz) δ 174.1 (C═O), 134.6, 121.0, 63.3, 52.9 (2C), 46.0 (3C), 34.1, 30.6, 29.1, 28.9 (2C), 24.7, 19.9, 8.7 (3C), 8.1 (2C); ¹⁹F NMR (CDCl₃, 282.6 MHz) δ −82.2 (m, 6F), −115.7 (m, 4F), −123.2 (m, 4F), −124.2 (m, 4F), −124.9 (m, 4F), −127.5 (m, 4F); ³¹P {1H}NMR (CDCl₃, 121 MHz) δ 5.65 (s); ESI-MS (spray voltage: 4.5 kV, capillary temp: 250° C., capillary voltage: 40 V, tube lens offset: 250 V) calcd. for [C₃₅H₄₂F₂₆O₇P]⁻ m/z 1099.6, found m/z 1099.3.

General Procedure for Compounds 1-5.

R−CH₂OH (6, 7 or 8, 0.12 mmol) (Liu et al., J. Am. Chem. Soc. 2006, 128, 3638-3648; Sharma et al., Polymer 2004, 45, 5427-5440 and H-phosphonate (12 or 13, 0.08 mmol) (Liu et al. J. Am. Chem. Soc. 2006, 128, 3638-3648), were co-evaporated with anhydrous pyridine (5 mL) and dried under high vacuum overnight. The resulting solution was dissolved in anhydrous pyridine (1 mL) at room temperature and pivaloyl chloride (20.4 μL, 0.16 mmol) was added to it. The reaction was stirred at rt for 10 h. Iodine (21.1 mg, 0.08 mmol) in a mixture of pyridine/water (19:1, 0.1 mL) was then added to the reaction mixture, and the reaction stirred for an additional 6 h at room temperature. The reaction mixture was diluted with CHCl₃ (50 mL) and washed with aqueous Na₂S₂O₃ (25 mL). The aqueous layer was extracted with CHCl₃ (3×50 mL). The combined organic layers were washed with TEAB buffer (50 mL), dried over MgSO₄ and solvents were removed under reduced pressure to afford crude product. TLC analysis showed a single spot that stained with Dragendorff's reagent. The product was purified by flash chromatography with Et₃N-deactivated silica gel to give 1-5 (72-85%) as pale yellowish gummy solids. R_{f} 0.50 (10% MeOH in CHCl₃).

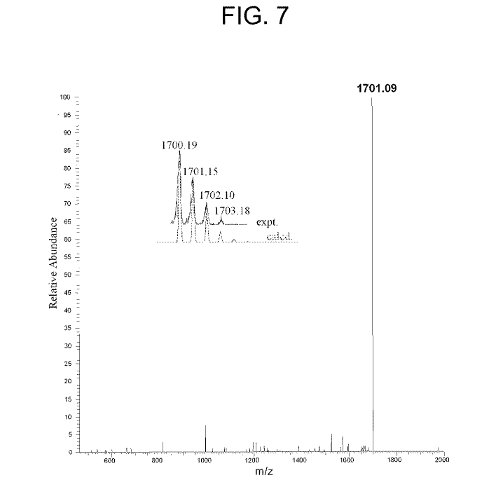

Compound 1. ¹H NMR (1:9 CD₃OD:CDCl₃, 300 MHz) δ 7.17 (m, 1H), 7.02 (m, 1H), 5.94 (s, 1H), 5.70 (s, 1H), 5.02 (m, 1H), 4.41 (m, 1H), 4.24 (m, 2H), 4.03 (m, 1H), 3.53 (t, J=5.7 Hz, 3H), 3.20 (m, 4H), 2.83 (dd, J=12.9 Hz, J=4.8 Hz, 1H), 2.64 (d, J=12.9 Hz, 1H), 2.48 (q, J=7.2 Hz, 6H), 2.25 (t, J=8.1 Hz, 4H), 2.10 (dd, J=14.1 Hz, J=8.1 Hz, 4H), 1.95 (m, 4H), 1.10-1.65 (m, 42H), 0.99 (t, J=7.2 Hz, 9H); ¹³C NMR (1:9 CD₃OD:CDCl₃, 75.5 MHz) δ 179.2, 179.1, 175.4. 174.1, 173.7, 70.5, 64.1, 63.5, 60.5, 58.2, 55.1, 46.4 (3C), 42.7, 41.6, 39.4, 38.9, 35.8, 34.7, 33.8, 34.4, 32.1, 31.5, 31.2, 30.9, 30.1 (2C), 30.0, 29.6 (2C), 29.4 (2C), 29.1, 28.7, 28.5, 28.3, 28.1, 27.7, 27.6 (2C), 27.0, 26.9, 26.5, 26.2, 25.8, 25.3, 25.1, 22.8, 20.4 (2C), 15.3, 14.5, 11.3, 9.1 (3C), 8.5 (2C); ¹⁹F NMR (1:9 CD₃OD:CDCl₃, 282.6 MHz) δ −81.3 (m, 6F), −114.9 (m, 4F), −122.5 (m, 4F), −123.4 (m, 4F), −124.1 (m, 4F), −126.7 (m, 4F); ³¹P {1H}NMR (1:9 CD₃OD:CDCl₃, 121 MHz) δ 0.95 (s); ESI-MS (spray voltage: 4.5 kV, capillary temp: 100° C., capillary voltage: 40 V, tube lens offset: 250 V) calcd. for [C₅₃H₇₂F₂₆N₄O₁₁PS.NEt₃H]⁺ m/z 1701.5, found m/z 1701.1. FIG. 7 is a positive mode ESI-MS spectrum and isotopic distribution of compound 1 in CH₂Cl₂. The inset shows calculated and experimental spectra.

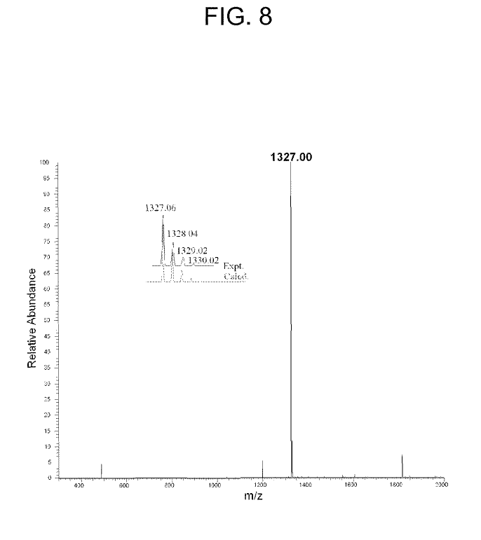

Compound 2. ¹H NMR (1:9 CD₃OD:CDCl₃, 300 MHz) δ 5.79 (s, 1H), 5.58 (s, 1H), 5.01 (m, 1H), 4.43 (m, 1H), 4.26 (m, 2H), 4.06 (m, 1H), 3.53 (m, 3H), 2.86 (dd, J=12.9 Hz, J=5.1 Hz, 1H), 2.66 (d, J=12.9 Hz, 1H), 2.48 (q, J=8.7 Hz, NEt₃H), 2.27 (t, J=7.8 Hz, 4H), 1.94 (m, 4H), 1.51 (m, 8H), 1.37 (m, 8H), 1.18 (m, 19H), 0.96 (t, J=7.2 Hz, NEt₃H), 0.89 (m, 4H); ¹³C NMR (1:9 CD₃OD:CDCl₃, 75.5 MHz) δ 184.2, 179.2, 178.1, 64.6, 64.5, 64.1, 64.3, 62.5, 60.8, 58.3, 55.7, 45.3 (3C), 44.3, 41.7, 40.8, 40.5, 39.3, 39.1, 34.2, 34.3, 31.7, 31.5, 31.2, 30.1, 29.7, 29.5, 29.4, 29.0, 28.9, 28.7, 28.3, 28.2, 27.7 (2C), 27.4, 27.2, 26.9, 26.8, 26.3, 26.2, 25.1, 24.5, 23.5, 20.4, 15.1, 14.4, 8.8 (3C); ¹⁹F NMR (1:9 CD₃OD:CDCl₃, 282.6 MHz) δ −81.3 (m, 6F), −114.9 (m, 4F), −122.5 (m, 4F), −123.4 (m, 4F), −124.1 (m, 4F), −126.7 (m, 4F); ³¹P {1H}NMR (1:9 CD₃OD:CDCl₃, 121 MHz) δ −0.57 (s); ESI-MS (spray voltage: 6.5 kV, capillary temp: 100° C., capillary voltage: 0 V, tube lens offset: −250 V) calcd. for [C₄₅H₅₈F₂₆N₂O₉PS]⁻ m/z 1327.3, found m/z 1327.0. FIG. 8 is a negative mode ESI-MS spectrum and isotopic distribution of compound 2 in CH₂Cl₂. The inset shows calculated and experimental spectra.

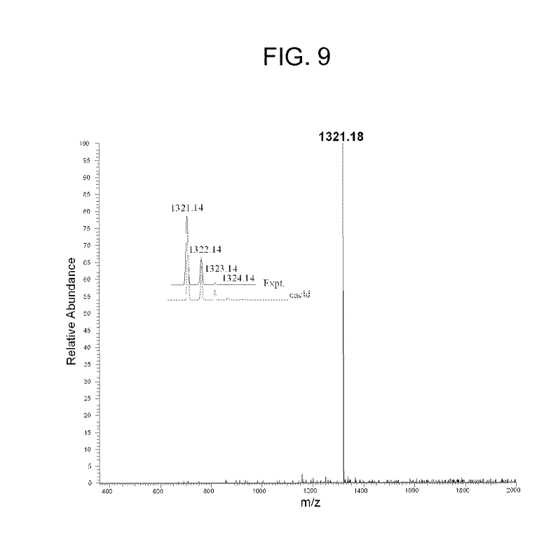

Compound 3. ¹H NMR (1:9 CD₃OD:CDCl₃, 300 MHz) δ 8.49 (d, J=8.7 Hz, 1H), 6.93 (s, 1H), 6.28 (d, J=8.41q Hz, 1H), 5.03 (m, 1H), 4.29 (m, 2H), 3.81 (m, 4H), 3.72 (m, 2H), 3.14 (q, J=7.2 Hz, NEt₃H), 2.32 (m, 4H), 2.25 (m, 4H), 1.60 (m, 8H), 1.46-1.11 (m, 29H); ¹³C NMR (CDCl₃, 75.5 MHz) δ 179.5 (2C), 144.7, 144.1, 136.7 (3C), 125.1, 99.4, 62.1 (4C), 46.4 (2C), 44.6, 43.5 (3C), 39.3 (2C), 35.2, 33.1, 31.5, 30.1, 29.6, 29.4, 27.5 (9C), 27.8, 22.3, 21.5, 17.4, 17.1, 10.7, 8.8 (3C), 9.0 (2C), 1.4, 1.2; ¹⁹F NMR (CDCl₃, 282.6 MHz) δ −81.3 (m, 6F), −114.9 (m, 4F), −122.5 (m, 4F), −123.4 (m, 4F), −124.1 (m, 4F), −126.6 (m, 4F); ³¹P {1H}NMR (CDCl₃, 121 MHz) δ −0.53 (s); ESI-MS (spray voltage: 6.5 kV, capillary temp: 100° C., capillary voltage: 0 V, tube lens offset: −250 V) calcd. for [C₄₃H₄₈F₂₆N₄O₁₁P]⁻ m/z 1321.3, found m/z 1321.2. FIG. 9 is a negative mode ESI-MS spectrum and isotopic distribution of compound 3 in CH₂Cl₂. The inset shows calculated and experimental spectra.

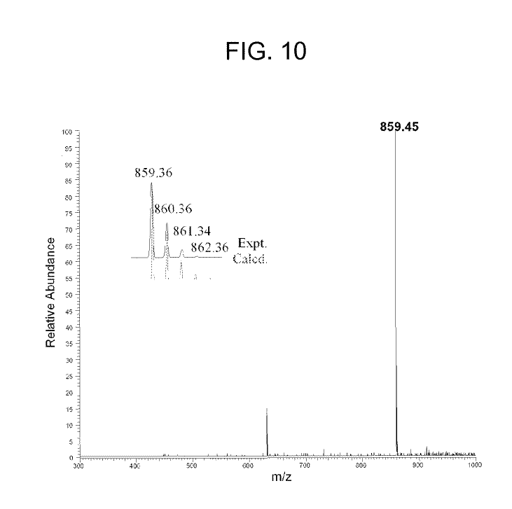

Compound 4. ¹H NMR (1:9 CD₃OD:CD₂Cl₂, 300 MHz) δ 5.11 (m, 1H), 4.51 (m, 1H), 4.40 (dd, J=12.0 Hz, J=3.6 Hz, 1H), 4.33 (dd, J=7.8 Hz, J=3.6 Hz, 1H), 4.20-4.04 (m, 3H), 3.58 (t, J=6.3 Hz, 2H), 3.20-3.26 (m, 1H), 2.95 (dd, J=12.6 H, J=4.8 Hz, 1H), 2.74 (m, J=12.6 Hz, 1H), 2.58 (q, J=7.2 Hz, NEt₃H), 2.39-2.32 (m, 6H), 1.73-1.31 (m, 58H), 1.07 (t, J=7.2 Hz, NEt₃H), 0.89 (m, 6H); ¹³C NMR (1:9 CD₃OD:CD₂Cl₂, 75.5 MHz) δ 174.5, 173.4, 171.5, 73.5, 72.1, 77.2, 65.1, 62.4, 61.9, 60.8, 57.5, 48.5 (3C), 46.3 (3C), 40.2, 34.3, 34.2, 34.1, 32.4, 32.2, 29.9 (6C), 29.7 (3C), 29.6, 29.5, 29.3, 29.2, 28.8, 25.9, 25.1, 22.9 (3C), 13.7 (3C), 9.4 (3C), 8.9 (3C); ³¹P {1H}NMR (1:9 CD₃OD:CD₂Cl₂, 121 MHz) δ 0.15 (s); ESI-MS (spray voltage: 6.5 kV, capillary temp: 100° C., capillary voltage: 0 V, tube lens offset: −250 V) calcd. for [C₄₅H₈₄N₂O₉PS]⁻ m/z 859.6, found m/z 859.5. FIG. 10 is a negative mode ESI-MS spectrum and isotopic distribution of compound 4 in CH₂Cl₂. The inset shows calculated and experimental spectra.

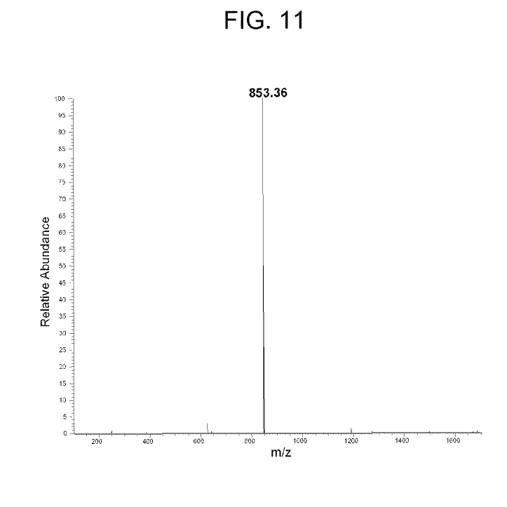

Compound 5. See K. Imai, Y. Tsukamoto, S. Uzu, S. Kanda, T. Toyooka, Y. Tachiiri, S. Fujiwake, Anal. Chim. Acta 1989, 223, 299. FIG. 11 is a negative mode ESI-MS spectrum of compoun 5 in CH₂Cl₂.

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A compound comprising a non-cationic phospholipid, a linker, and an agent for delivery to a cell, wherein at least one hydrocarbon chain of the phospholipid is fluorinated, and wherein the phospholipid, the linker, and the agent are covalently linked.

2. The compound of claim 1, wherein the compound is of the formula: 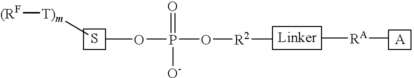
wherein: 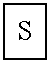
is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{A} is a covalent bond or an optionally substituted moiety derived from conjugating an optionally substituted thiol-reactive, amine-reactive, or hydroxyl-reactive moiety with a thiol, amine, or hydroxyl group of the agent; 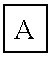
is a therapeutic agent;
each occurrence of n is an integer from 0 to 30, inclusive, wherein at least one occurrence of n is non-zero; and
m is an integer from 1 to 2, inclusive, wherein m is 1 when 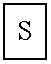
is a covalent bond.

3. A compound comprising a non-cationic phospholipid, wherein at least one hydrocarbon chain of the phospholipid is fluorinated and the compound is of the formula: 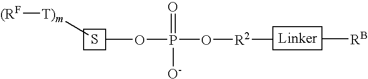
wherein: 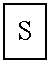
is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{B} is an optionally substituted moiety capable of forming a non-covalent interaction with a therapeutic agent;
each occurrence of n is an integer from 0 to 30, inclusive, wherein at least one occurrence of n is non-zero; and
m is an integer from 1 to 2, inclusive, wherein m is 1 when 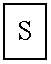
is a covalent bond.

4. A composition comprising:
a) a non-cationic phospholipid of claim 3, and
b) a therapeutic agent 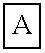
non-covalently linked to R^{B}.

5-6. (canceled)

7. The compound of claim 1, wherein the linker is a biodegradable linker.

8. The compound of claim 7, wherein the linker is enzyme sensitive.

9. The compound of claim 8, wherein the linker is beta-glucosidase sensitive, calpain sensitive, carboxyesterase sensitive, or lysosomal aminopeptidase sensitive.

10. The compound of claim 9, wherein the linker is selected from the group consisting of: 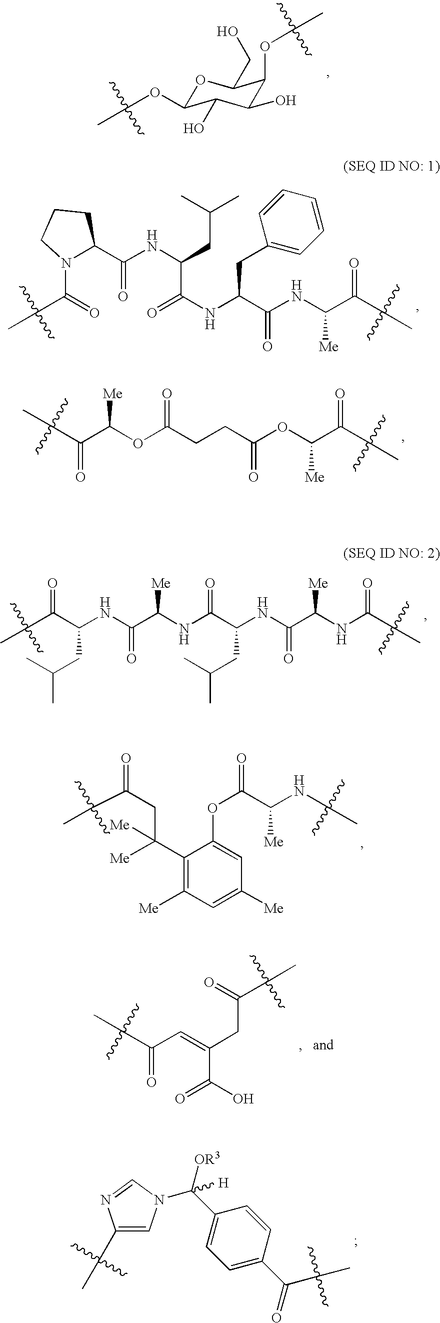
wherein R³ is C₁₋₆ aliphatic.

11-17. (canceled)

18. The compound of claim 1, wherein the linker is cleaved under acidic conditions.

19-24. (canceled)

25. The compound of claim 2, wherein 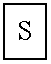
is selected from: 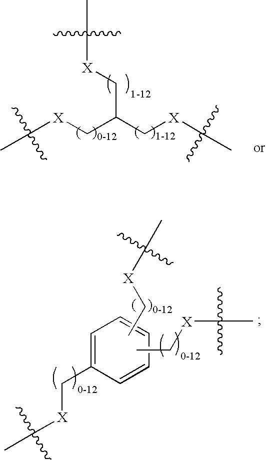
wherein X is N, O, or S.

26-28. (canceled)

29. The compound of claim 2, wherein T is a moiety selected from the group consisting of —CⁿH_{2n′}C(O)—, —CₙH_{2n′}OC(O)—, and —CₙH_{2n′}N(R)C(O)—; wherien n′ is an integer from 1 to 28, inclusive.

30-31. (canceled)

32. The compound of claim 2, wherein n is an integer from 1 to 20, inclusive.

33-36. (canceled)

37. The compound of claim 2, wherein R² is selected from the group consisting of:
—C₂H₄NH—, 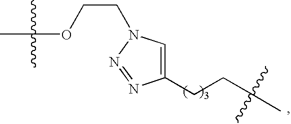
and is 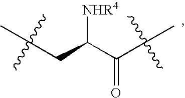
wherein R⁴ is hydrogen or a protecting group.

38-40. (canceled)

41. The compound of claim 2, wherein R^{A} is an optionally substituted moiety derived from conjugating an optionally substituted thiol-reactive, amine-reactive, or hydroxyl-reactive moiety with a thiol, amine, or hydroxyl group of the agent.

42. The compound of claim 41, wherein R^{A} is selected from the group consisting of: 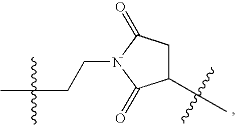
—C(O)—, —CH₂— and —CH₂C(O)—.

43-45. (canceled)

46. A non-cationic fluorinated phospholipid of the formula: 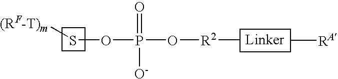
wherein: 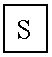
is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{A′} is hydrogen or an optionally substituted thiol-reactive, amine-reactive, or hydroxyl-reactive moiety;
each occurrence of n is an integer from 0 to 30, inclusive, wherein at least one occurrence of n is non-zero; and 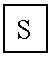
m is an integer from 1 to 2, inclusive, wherein m is 1 when is a covalent bond.

47-48. (canceled)

49. The compound of claim 1, wherein the agent for delivery to a cell comprises a macromolecule, a small molecule, or a flurophore.

50. The compound of claim 49, wherein the macromolecule comprises a nucleic acid.

51-53. (canceled)

54. The compound of claim 50, wherein the nucleic acid comprises RNA.

55-63. (canceled)

64. The compound of claim 1, further comprising a pharmaceutically acceptable carrier.

65. A method for delivering a therapeutic agent into a cell, the method comprising contacting a cell with a composition comprising the compound of claim 1.

66-69. (canceled)

70. A method comprising steps of:
delivering parenterally to a subject a composition comprising a compound as recited in claim 1.

71-72. (canceled)

73. A kit for delivering a macromolecule into a cell, the kit comprising a compound of claim 1.

74-80. (canceled)

81. A method comprising the steps of:
a) providing a non-cationic fluorinated phospholipid of the formula: 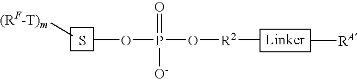
wherein: 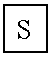
is a covalent bond or an optionally substituted group selected from the group consisting of acyl, aliphatic, heteroaliphatic, aryl, heteroaryl, and heterocyclic;
each occurrence of T is independently a covalent bond or a bivalent, straight or branched, saturated or unsaturated, C₁₋₄₀ hydrocarbon chain wherein one or more methylene units of T are optionally and independently replaced by —CF₂—, —O—, —S—, —N(R)—, —C(O)—, —C(O)O—, —OC(O)—, —N(R)C(O)—, —C(O)N(R)—, —S(O)—, —S(O)₂—, —N(R)SO₂—, or —SO₂N(R)—;
each occurrence of R is independently hydrogen, a protecting group, or an acyl moiety, arylalkyl moiety, aliphatic moiety, aryl moiety, heteroaryl moiety, or heteroaliphatic moiety; or: two R on the same nitrogen atom are taken with the nitrogen to form a 4-7-membered heterocyclic ring having 1-2 heteroatoms independently selected from nitrogen, oxygen, or sulfur;
each occurrence of R^{F} is a group having the formula —CₙF₍₂ₙ₊₁₎;
R² is a covalent bond or an optionally substituted bivalent, straight or branched, saturated or unsaturated, C₁₋₂₀ aliphatic or C₁₋₂₀ heteroaliphatic chain, wherein one or two methylene units are optionally and independently replaced by an optionally substituted group selected from 6-10 membered aryl, 5-10 membered heteroaryl having 1-4 heteroatoms independently selected from nitrogen, oxygen, or sulfur, and 4-7 membered heterocyclyl having 1-2 heteroatoms independently selected from nitrogen, oxygen, or sulfur;
the linker is a peptide, an optionally substituted bivalent moiety selected from the group consisting of acyl, aliphatic, heteroaliphatic, aryl, heteroaryl, and heterocyclic;
R^{A′} is hydrogen or an optionally substituted thiol-reactive, amine-reactive, or hydroxyl-reactive moiety;
each occurrence of n is an integer from 0 to 30, inclusive, wherein at least one occurrence of n is non-zero; and
m is an integer from 1 to 2, inclusive, wherein m is 1 when 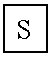
is a covalent bond; and
b) contacting the fluorinated phospholipid with an agent for delivery to a cell to form a compound of formula: 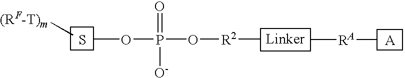
wherein:
R^{A} is a covalent bond or an optionally substituted moiety derived from conjugating an optionally substituted thiol-reactive, amine-reactive, or hydroxyl-reactive moiety with a thiol, amine, or hydroxyl group of the agent; and 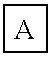
is a therapeutic agent.

82-84. (canceled)

85. The compound of claim 2, wherein the compound is of the formula: 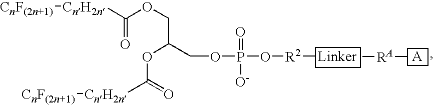
wherein n′ is an integer from 1 to 28, inclusive.

86. The compound of claim 85, wherein the compound is of the formula:

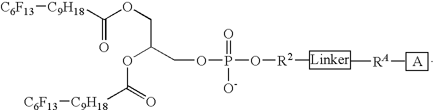

87. The compound of claim 86, wherein the linker is an optionally substituted bivalent aryl moiety.

88. The compound of claim 86, wherein the linker comprises a cleavable linkage.

89. The compound of claim 88, wherein the cleavable linkage includes an acetal bond.

90. The compound of claim 86, wherein R² is —C₂H₄NH—.

91. The compound of claim 86, wherein R² is an optionally substituted bivalent C₁₋₂₀ heteroaliphatic chain, wherein one methylene unit is replaced by an optionally substituted 6-10 membered aryl group.

92. The compound of claim 91, wherein the linker comprises a cleavable linkage and the cleavable linkage includes an acetal bond.

93. The compound of claim 86, wherein R^{A} is an optionally substituted moiety derived from a cross linking reagent capable of conjugating an amine or hydroxyl of the linker with a thiol or amine of the therapeutic agent.

94. The compound of claim 93, wherein R^{A} is derived from a cross linking agent comprising a maleimide.

95. The compound of claim 46, wherein R^{A′} is an optionally substituted thiol-reactive moiety.

96. The compound of claim 46, wherein R^{A′} is an optionally substituted amine-reactive moiety.


SEQUENCE LISTING (2 sequences)

SEQ ID NO: 1 - protein, 4 aa, Artificial Sequence
  Calpain sensitive sequence
PLFA

SEQ ID NO: 2 - protein, 4 aa, Artificial Sequence
  Lysosomal aminopeptidase sensitive sequence
VAVA